SEQUENCE LISTING (53 sequences)

SEQ ID NO: 1 - DNA, 1887 bp, Arabidopsis thaliana
  CDS (1)..(1884)
atggctggtggagctatcactcccgattctctcatcggtcttattgctgaaatcaatgagattccggggaattttggtttgtttaagaaggattgttccgatctcgcgcgacgagtcggtctcctgacgcatttgattgaggaaatcagggattcttctccgccgtcggaatctgatgcttcttcttcgttgaattctcatgaatgtgattggtggtctgatcttgtggtgggacttcaagccgcgaagcgtcttttgtcttcagctactagtttccaagctcgcgagtcctctgatggggcagccaagaggatctcattccagttccaatgtgttacatggaagctggagaaagcattaggtgatttgacatatgatagatatgatatctctgacgaagtccgtgaacaggtggaactagcaagattacagttaagaagagcaatgcagagatatggatcgttaaattcgaagaagttctcgagtggtttatctgagccaatggagaaagatgcctcaagtaatagaaaagttattgaaaagttggagagtattccagagacagtgcattctttatcagatgagaagaaatttgaatcaccccctccttggaagagctcttcagtatcattggccttctttttatcaaaggatggtgatgatgagagattagagaaagcagttaccgagaacagcgatgactcacagaaatcggataatctcacgattccagaggactttctttgtccaatctctctggaactgatgaaggatcctgctattgtttccacaggacagacatacgagaggtcgttcattcagagatggatagactgtggaaatctgagctgcccaaagacacagcagaaacttgaaaactttactcttactccaaactatgtcctcagaagcctgatatctcagtggtgcactaagcacaacattgagcaaccaggtggttacatgaacggtaggaccaaaaacagtgatgggtctttccgtgatctaagtggagatatgtcagcgatccgagcattggtttgtaaactctctagccaatcaatcgaagaccgcaggactgcggtttctgaaatccgctctctgtcaaaaagaagcacagacaaccgaattctgattgcagaggcaggagccatccctgttttggtcaagcttttgacctcagatggtgacactgaaacgcaggaaaatgctgtcacttgcattcttaacctctccatatacgaacacaacaaagaactgatcatgcttgcaggagcagtcacatctatagtgctagtcctccgagctggaagcatggaagctagagaaaacgctgcagctactctctttagtctttcgttagctgatgagaacaagatcattataggcgcatcaggtgcgataatggccttagtggatctgcttcaatatggaagcgtcagagggaagaaagacgcagctacggctctgttcaacttgtgtatttaccagggaaacaaaggcagagcagttagagccggtatagtaaagccattggtgaaaatgctgacagactcctcgagcgagaggatggctgatgaagccttgaccatactctcggttctagctagtaaccaagtcgcgaaaactgcgattttgagagccaacgcaataccacctttgatagattgtctccagaaggatcaaccgcgaaaccgagagaacgcagctgcgatactactgtgtctttgcaagagagatactgaaaaactgatctccattggtagacttggagctgttgttccattgatggaactatcaagagacggtacagagagagctaaacgaaaagctaattctttactagagcttctccgtaaatcatcccgaaaattaggttcactctaa

SEQ ID NO: 2 - protein, 628 aa, Arabidopsis thaliana
MAGGAITPDSLIGLIAEINEIPGNFGLFKKDCSDLARRVGLLTHLIEEIRDSSPPSESDASSSLNSHECDWWSDLVVGLQAAKRLLSSATSFQARESSDGAAKRISFQFQCVTWKLEKALGDLTYDRYDISDEVREQVELARLQLRRAMQRYGSLNSKKFSSGLSEPMEKDASSNRKVIEKLESIPETVHSLSDEKKFESPPPWKSSSVSLAFFLSKDGDDERLEKAVTENSDDSQKSDNLTIPEDFLCPISLELMKDPAIVSTGQTYERSFIQRWIDCGNLSCPKTQQKLENFTLTPNYVLRSLISQWCTKHNIEQPGGYMNGRTKNSDGSFRDLSGDMSAIRALVCKLSSQSIEDRRTAVSEIRSLSKRSTDNRILIAEAGAIPVLVKLLTSDGDTETQENAVTCILNLSIYEHNKELIMLAGAVTSIVLVLRAGSMEARENAAATLFSLSLADENKIIIGASGAIMALVDLLQYGSVRGKKDAATALFNLCIYQGNKGRAVRAGIVKPLVKMLTDSSSERMADEALTILSVLASNQVAKTAILRANAIPPLIDCLQKDQPRNRENAAAILLCLCKRDTEKLISIGRLGAVVPLMELSRDGTERAKRKANSLLELLRKSSRKLGSL

SEQ ID NO: 3 - DNA, 1839 bp, Arabidopsis thaliana
  CDS (1)..(1836)
atggccggaggaatcgtctcaccggcctctctactcgacctaatcgctgatatcgtcgagattccactcaacaccggaatgttcaagaaggattgcgcagatctcactcgacgagtttgtctcttaacgcatttgttagaggagattagagactcaactcctatcgattccgccgcttcgtcgtcgtcggagaatgattggtggtctgatctcgtggtcggacttcaagccgcgaagcgtcttctctccacagctcgattccaagctcgcgattcctctgatggtgctgcgaagagaatctcatttcagttccaatgtgttacatggaagcttgagaaagcattgagcaatcttccatatgatctttatgatatctctgacgaagttggagaacaagtggagctagcgagatcacagttgcgaagagcaatgcagagatatggatcgttgaattcgaacaagttctcgagtgctttatctgagccaatggagagagatggttttagtaatgttataaagatcaaagctgaggaaaagcttgagagtgtttcagaaacacttcattttggtgaggaggaggagaaacaaagttcgccgcctctaaggagaagctcttccatttctttggcttattatttgtccaaagatgctgatactgatagattagacaaaatggttaacaagaacactgatgaatcgaagaaatctgataaactcacgattccggttgattttctttgtccggtgtcgcttgaactgatgaaggatcctgtcattgttgccacaggacagacttacgagagggcatacatacagagatggatcgactgtgggaatctaacatgtcctaagactcagcagaaactcgaaaactttacgcttacaccaaactatgttctcagaagtctcatctctcggtggtgcgctgaacacaacattgagcaacctgcaggttacattaacggcaggacaaaaaacagtggagatatgtcggtgatccgggccttggttcagagactctcaagccggtcaacagaggatcgaaggaatgcggtttctgaaatccgctctctgtcaaagagaagtacggataatcgcattctgattgcagaagcaggagcaatccctgttttggtgaatcttttaacctcagaggatgttgcaacgcaggaaaacgcaatcacatgtgttctcaacctctctatatatgaaaacaacaaagagctgataatgtttgctggtgcagtcacctcaattgttcaagtccttagagccggaaccatggaagcaagagaaaacgcagcagcgacactttttagcctgtcgttagccgatgagaacaagatcataataggcgggtcaggtgcgatacctgccttagttgatctgctcgagaacgggaccccgagagggaagaaagatgcagcaacggcattgttcaatctctgcatttatcatggaaacaaaggtagagcagtcagagccggtatagtaactgcattggttaagatgctcagtgattccacccgccaccggatggttgatgaagctttgacaattctctcagttcttgcaaataatcaagacgcgaaatccgcgatagtaaaagcgaatactctacccgcgttgataggtattctccaaacggatcaaactagaaaccgagagaacgcagcagcgatattgctttcgctgtgtaaaagagacactgagaaactgatcactatcggtagactcggtgcggttgtcccgttaatggatttatcgaagaatggaacagagagaggcaaaaggaaagccatatctttgttagagcttcttcgtaaagcatgccaataa

SEQ ID NO: 4 - protein, 612 aa, Arabidopsis thaliana
MAGGIVSPASLLDLIADIVEIPLNTGMFKKDCADLTRRVCLLTHLLEEIRDSTPIDSAASSSSENDWWSDLVVGLQAAKRLLSTARFQARDSSDGAAKRISFQFQCVTWKLEKALSNLPYDLYDISDEVGEQVELARSQLRRAMQRYGSLNSNKFSSALSEPMERDGFSNVIKIKAEEKLESVSETLHFGEEEEKQSSPPLRRSSSISLAYYLSKDADTDRLDKMVNKNTDESKKSDKLTIPVDFLCPVSLELMKDPVIVATGQTYERAYIQRWIDCGNLTCPKTQQKLENFTLTPNYVLRSLISRWCAEHNIEQPAGYINGRTKNSGDMSVIRALVQRLSSRSTEDRRNAVSEIRSLSKRSTDNRILIAEAGAIPVLVNLLTSEDVATQENAITCVLNLSIYENNKELIMFAGAVTSIVQVLRAGTMEARENAAATLFSLSLADENKIIIGGSGAIPALVDLLENGTPRGKKDAATALFNLCIYHGNKGRAVRAGIVTALVKMLSDSTRHRMVDEALTILSVLANNQDAKSAIVKANTLPALIGILQTDQTRNRENAAAILLSLCKRDTEKLITIGRLGAVVPLMDLSKNGTERGKRKAISLLELLRKACQ

SEQ ID NO: 5 - DNA, 1872 bp, Arabidopsis thaliana
  CDS (1)..(1869)
atgactgattaccggctacaaccaacgatgaatctttggaccaccgacgacaacgcttctatgatggaagctttcatgagctcttccgatatctcaactttatggcctccggcgtcgacgacaaccacgacggcgacgactgaaacaactccgacgccggcgatggagattccggcacaggcgggatttaatcaagagactcttcagcaacgtttacaagctttgattgaaggaacacacgaaggttggacctacgctatattctggcaaccgtcgtatgatttctccggcgcctccgtgctcggatggggagatggttattacaaaggtgaagaagataaagcaaacccgagacggagatcgagttcgccgccgttttctactccggcggatcaggagtacaggaaaaaagtgttgagagagcttaactcgttgatctccggtggtgttgctccgtcggatgacgctgttgatgaggaggtgacggatacggaatggtttttcttggtttcgatgacgcagagcttcgcttgcggtgcgggattagctggtaaagcgtttgcaacgggtaacgcggtttgggtttccgggtcagatcaattatccgggtcgggttgtgaacgggctaagcaaggaggagtgtttgggatgcatactattgcgtgtattccttcggcgaacggagttgtggaagtcgggtcaacggagccgatccgacagagttcggaccttattaacaaggttcgaattcttttcaatttcgacggcggagctggagatttatcgggtcttaattggaatcttgacccggatcaaggtgagaacgacccgtctatgtggattaatgacccgattggaacacctggatctaacgaaccgggtaacggagctccaagttctagctcccagcttttttcaaagtctattcagtttgagaacggtagctcaagcacaataaccgaaaacccgaatctggatccgactccgagtccggttcattctcagacccagaatccgaaattcaataacactttctcccgagaacttaatttttcgacgtcaagttctactttagtgaaaccaagatccggcgagatattaaacttcggcgatgaaggtaaacgaagctccggaaacccggatccaagttcttattcgggtcaaacacaattcgaaaacaaaagaaagaggtcgatggttttgaacgaagataaagttctatcattcggagataaaaccgccggagaatcagatcactccgatctagaagcttccgtcgtgaaagaagtagcagtagagaaacgtccaaagaaacgaggaagaaagccagcaaacggtagagaagagccactaaaccacgtcgaagcagagagacaaagacgcgagaaactaaaccaaagattctacgcgttacgagcggttgtaccaaacgtttcaaaaatggataaagcttcgttactcggtgacgcaatcgcttacatcaacgagcttaaatccaaagtagtcaaaacagagtcagagaaactccaaatcaagaaccagctcgaggaagtgaaactcgagctcgccggaagaaaagcgagtgctagtggaggagatatgtcgtcttcgtgttcttcgattaaaccggtggggatggagattgaagtgaagataattggttgggacgcaatgattagagttgaatctagtaagaggaatcatccggcggcgaggttgatgtcggcgttgatggatttggagttggaagtgaatcacgcgagtatgtcggtggttaacgatttgatgattcaacaagcgacggtgaagatgggttttaggatctatacgcaagaacagctcagagcaagtttgatttcaaaaatcggttaa

SEQ ID NO: 6 - protein, 623 aa, Arabidopsis thaliana
MTDYRLQPTMNLWTTDDNASMMEAFMSSSDISTLWPPASTTTTTATTETTPTPAMEIPAQAGFNQETLQQRLQALIEGTHEGWTYAIFWQPSYDFSGASVLGWGDGYYKGEEDKANPRRRSSSPPFSTPADQEYRKKVLRELNSLISGGVAPSDDAVDEEVTDTEWFFLVSMTQSFACGAGLAGKAFATGNAVWVSGSDQLSGSGCERAKQGGVFGMHTIACIPSANGVVEVGSTEPIRQSSDLINKVRILFNFDGGAGDLSGLNWNLDPDQGENDPSMWINDPIGTPGSNEPGNGAPSSSSQLFSKSIQFENGSSSTITENPNLDPTPSPVHSQTQNPKFNNTFSRELNFSTSSSTLVKPRSGEILNFGDEGKRSSGNPDPSSYSGQTQFENKRKRSMVLNEDKVLSFGDKTAGESDHSDLEASVVKEVAVEKRPKKRGRKPANGREEPLNHVEAERQRREKLNQRFYALRAVVPNVSKMDKASLLGDAIAYINELKSKVVKTESEKLQIKNQLEEVKLELAGRKASASGGDMSSSCSSIKPVGMEIEVKIIGWDAMIRVESSKRNHPAARLMSALMDLELEVNHASMSVVNDLMIQQATVKMGFRIYTQEQLRASLISKIG

SEQ ID NO: 7 - DNA, 1577 bp, Arabidopsis thaliana
gagattgtttgctaagaaaatggaccaatatttatcatcgaaactacaatcatcattaaggttgtttctacgattgcgggatctacaaccatcaaagaagttgtttccacaactgtataaactctgatctcaccttcactttcatattgttccgcaagtacaactacaccataaatttcagaaataacggtgagtattgtggttaatggagagttgtgtgtggatgtgcaagcaaaaatcacatgcctcatcaaacacttcaaggttattaatcttcatctgattcagacgaataccctcttctttatgtagatctttttatttttgcatatgtccaaaaataattatgaaaccagccagtcaaacttgccaaaaatgtctaaaagataatgttgtctatttttcaagttgctatcggttgttatgtaggacgcagaagtcacgggcatggacgtcggccgatatgatgtggatgtcgtggttgaacctttcttttgttaaaccgagataacttttcaatacaagactgtcacaaactatatgtaagtcatgtttataaattttaatcaaaagtatactaaactgtggtaaaacagttttaagaaaattataggtcccaatttgtatacatgcttcaaattatctaaaatgttgttaaaattcgatctaccgggtcatgttcgaccgttccatttgcaggctatataatactcaccaaaattatgttaattgacataatttttctagaacatttttgctgtaagagatattaaattttcttacacatacaaagttatttttaagggaaaataaaacatgtaaagctatatttagttaattacaaagaagattttttccatcttttaataaacactagggtaagacagatcatatactagaaacacatgtcaatacaatgcatctaaaggcctggaaattttatcttagtttcttttcaaatagaaaaaataactaatttgactatttattaccatttaagattacataaataatacatacagataaatttcataactccaacataaatttcaacacaactataaaatacaatcatataagatctactttacaactaagcacacaacaaaccattttcttcaataaatagaaaagtggatttttttattgaatttaaaagtatagttcatttctattatacagttttgatctaatataaatgataataattgattatttttttgacaactgtatcattttctcttcaacctctacttgcaccgtgaatctcgctctcaattatctagtttgttctattgtattatttaaatgtatcacataaacttttatctaaaaatataaagccagtaaactaaaaagaaaagaaaaaaccatttttagagttagataaaaagtttaaaacaaaaaacactaaaaccaaaacggtgtcgtaacagcgacttatcctttttcgccacatggcttcgttggatacataaaagcgatagagacgcatctctgagtcagaagcgagccagcgagagagagagagcgagtcacaaaaattcgaattttaagccgccgtgtgagacggcgta

SEQ ID NO: 8 - DNA, 1604 bp, Arabidopsis thaliana
ctctaccctccagtttctagctccacgaccacattctatttgtgatccactttcattctaacaaaccaataatttgagtgcatttttaattcttaacccattttttgtaattctcacatgtcacttgcaccaaagagctagaagaactctcttatgatgttgataattccaccaacttggtgctgagttctttctcaccattggacgagctccatgataacgaattaggtttccatttcatagaggctcttataccatgtaaacttcgaaacatttagagaacttgttattgtataacacacgtacacaatgctataagtcgttataccatatttggccgttccagtaaaaacatttttgaatggttttgctgatgatgccgtatactgaacgtggacgatcatttttcctcctatcaaaaacagtaaataattctttaaccaaaaaaaagcatagtaaggaatttcatgataaatttacagaaaccgtagaagaatatataccaatcaataattggtgatgataacgtcaaaaaaaataaaaattgatgatgataatcttttaaatcaccataattgtttccggccggaaaaaaacagataactaatttttaacattgtggtccgaatatttattagattgacccattctgtttatatatattattaaaacccattacaactttcttttacataaaaagagatgataatataaagtcggctttacaacgaagcaaagaaagaaaatgtaatggtgctcgatcactcttatgtgtataattaaaagcgtgtgtgtggttcatttcaattatatgtgtttggttgcattatgaaccataatattccatagccaactgtatcctccttgttttgttctcttcttctcctacatctaccaccgccaatctcgccttttttttccctctcatttatttattaatctactcattttttttttttgacattatctactcagtcctttattttattaataatccaatatagaaatttaaaataagttaattctttcaaaacttagaaaaatacatgattgtcggatttttttatttgtcaataacatacaagagtttgaatccgaatagcaacataacaaaattactagcacaaaccattatacaataatgtcggttttctaattttaattttatttgtcagatttttttcgtttttgacattgttttatattttttctaggtattaaagtttagatgttggatttataacatagtttttaggatttagatttagaatttaaactttgattattttgtcttataaaaatatagactaagactatttaatttcttaatgacttttcttcttttcgagactattccttaattatgatttgaaatatacactaaaaaaaaaagttgacaacaacaaaaaaagtggaccaagacgtaggtccttaaaacggtgccgtaacaaggacctctcgcttgacaaaatggtcgcgtgaataaataaaagcaacgttagtcgcttctgagtaaagaaccaatcccaaagctcatttattctttccttttcaattattctttcaatttttccgatcgacggaggttgatcggcgacggcgta

SEQ ID NO: 9 - DNA, 1592 bp, Arabidopsis thaliana
ctagtggcgtcacccccaaagtcgacatgaaatatgatatcacgaatcacctagccaaaacttagtgtaacatcttctcataattttcttatagtaaacatgttcaaaagtcgactcacaaccaccagacaatggtttggttgttttctgtttgttgaatcgttcattgacgagttcacccaatacagatgtctcagaaaattatattcaaaagttattttggttgttatttacaaaaaatggtatactagatcccaaaattattttactatttattgttggtaaaatactctttagtttaatatggttttaataagaaaaatcaaccggagtatcattaatctgaacgtataaagacatatcattgagtttatcacaagacaacgtaaatctacaagaacacaataacataagtttagtgttttgttagtaagaatttgtcttcgcaagatagaattttagaagtatatatatatatatatatatatactattttatcttaattctttttgttttatttttggtttttaggtaagggttaggagtaatgggaccatattggtggcatgctaaattattgtgataaatttatgcatggaggaaaagatgccaaatgatttatgttattcactcatactcacctaatgacctaatccttgaattattgttcgaactttaaaaataaaattttaatattgtaaattaaacaaacaatataacgttgttagcatgtgtttatcgtgaaatgtgaatgtaactaaaaatattataaaggagttcttcctttcttaaagcatatgctaataatagcgtttggtcaaattaataaattattagtttatttagtcgtcaacaaattaattagttaagcagacaagaatcgtttttggagatagagattctcacttgcatttcactctcttgcaatttaacatattaagacaaacacaaatttttgctttagtgaattattagcaacgactcacgttatatagatatgcaaatctcaaaattaattaagctaagacacagtatttgataattgagattacaaatcttaaattttgatttttaatattttaaaataacattataaaataagtaattaatctgttataaaaaatatattctaaaagttgacaaaacgacaaatataaatcgaaaaaataggtgaaaagttatagtaaagtgtttttccactgagctcacagttcactctttggtacaaaagaaacaatcagttaaaatctaagtcaatcattaaagagcgtgtgatacacgtgcgaccaaacagccgtacgatctcactcacttccggtcttaattcttaccgcgtcgatccaacagtcgatggggttaaatcgggatgtgagttcatcgaagcttctctctctcttacaaccatccacgtttcccaatgaaatctcgccacgtaatatccctaacaacaaatttacacattcaccctcaagatcagttacacatataataatcgcagacgctctccatttttctccactacgaagactttctcctatctctctctctctcattaaaaacgtgtttttttttaccggtcaccggtttatgga

SEQ ID NO: 10 - DNA, 459 bp, Arabidopsis thaliana
atgtcgacgccagcaaggaagaggttaatgagggatttcaagaggttgcagcaagacccacctgcgggtattagtggtgctccacaggacaacaacattatgctctggaatgctgtcatatttgggcctgatgacacaccatgggatggaggtactttcaaactctcactgcagttctctgaagattatcccaataaaccaccaacagttcggtttgtgtcacggatgtttcatcctaatatttatgcagatgggagtatctgcttggacattctacaaaaccagtggagtccaatctatgatgttgctgctatacttacctccatccagtccttgctctgtgaccctaatccgaattctcctgcaaactcggaagctgctcggatgtacagcgaaagcaagcgcgagtacaacaggagagtgcgtgatgttgttgagcaaagctggactgctgactag

SEQ ID NO: 11 - DNA, 459 bp, Arabidopsis thaliana
atgtcgactccagcgaggaagagattgatgagggatttcaagaggttgcagcaagacccacctgcaggaattagtggtgctccacaagacaacaatatcatgttgtggaatgctgttatattcgggcctgatgataccccttgggatggaggtactttcaaattgtcactgcagttttcggaagattatccaaataaaccaccaacagtgcggtttgtttcacggatgttccatccaaacatttatgctgatgggagtatatgcttggacattctccaaaaccaatggagtccaatatacgatgtcgctgctatactaacctccattcagtcattgctatgtgatcctaatccgaattctcctgcaaattcggaagctgcacgaatgttcagtgaaagcaagcgcgagtacaacagaagagtccgcgaggttgtcgaacaaagctggactgccgactag

SEQ ID NO: 12 - DNA, 453 bp, Arabidopsis thaliana
atgacaactccagcaaaaaagagactgatgtgggatttcaagagactgcagaaagatcctcctgtgggaataagtggagctccacaagataacaatatcatgcattggaatgctctcatctttggacctgaggacactccatgggatggaggtactttcaagttgactcttcacttcaccgaggattatccgaacaaacccccgattgttcggtttgtttcacggatgtttcatccaaacatttatgctgatggaagcatttgcttggatatcttgcaaaaccagtggagccccatctacgacgttgctgcagttctcacatcaattcagtctttgctctgtgacccaaaccctgactcaccagcgaacgcagaggctgcaaggttgttcagtgagaataaaagagaatataacaggaaagtaatcgagatcgtggagcagagctatgtataa

SEQ ID NO: 13 - DNA, 558 bp, Arabidopsis thaliana
atgtcttcgccgagcaagcgtagagaaatggatttgatgaaactgatgatgagtgactacaaggtggagatgatcaatgatggcatgcaagagttctttgtcgaattcagtggacccaaagacagtatatatgagggaggtgtgtggaagataagagttgaacttcctgatgcttatccttataaatctccatctgttggtttcattaccaaaatataccaccctaatgtcgatgaaatgtcaggttctgtttgtctagatgttattaaccagacctggagcccgatgttcgacctggtgaatgtgttcgagacatttcttccgcaacttcttctgtatccgaatccgtcagatccattgaatggtgaagcagctgcattgatgatgcgtgatcgtcctacctatgaacagagagttaaagagtattgtgagaaatacgcaaagccaagggcggatactgaggaaatgtctagtgatgatgaaatgagtgaagatgaatatgcctcggatggtgatgatgaagatgatgttgcaatcgccggtaaacttgatccttaa

SEQ ID NO: 14 - DNA, 447 bp, Arabidopsis thaliana
atggcttcgaaacggatcttgaaggagctgaaggatctccagaaagaccctccaacctcctgcagtgcaggtccagttgctgaagacatgtttcattggcaagctacaattatgggtcctgcagagagtccgtattcaggcggtgtgtttctcgttaccattcacttccctccggactatccattcaaaccaccaaaggttgcatttaggacgaaggtgtttcaccctaatatcaacagcaacggaagcatttgccttgacattttgaaagaacaatggagccctgccctcaccatttccaaggtgttgctctcgatatgttcgctgttaacagatccaaatccagatgaccctttggtaccagagattgcacacatgtacaaaaccgacagagccaaatacgaggctactgcaagaaactggactcagaagtatgccatgggctaa

SEQ ID NO: 15 - DNA, 447 bp, Arabidopsis thaliana
atggcgtcgaagcggatcttgaaggaattgaaggatctccagaaggatccacctacatcatgtagcgcaggtcctgttgctgaagacatgtttcactggcaggcaacgataatgggtccttcagagagtccttatgctggaggtgttttccttgtaaccatccatttccctccggattacccattcaagcctcctaaggtggcctttaggaccaaggtgttccatcccaacattaacagcaacggtagcatttgcctcgacatcttgaaggagcagtggagtcctgcgctcaccatttccaaggtgctgctatcgatctgttctttgttaaccgatccaaacccggatgatcctttggtgcccgagatagctcacatgtacaagactgacaagaacaagtacgagtccactgcacgaagctggactcagaagtatgccatgggctaa

SEQ ID NO: 16 - DNA, 447 bp, Arabidopsis thaliana
atggcttctaagaggatcttgaaggagctcaaggatctgcagaaggatcctccttcaaactgcagcgctggtccggtggctgaggacatgttccattggcaagccactatcatgggacctcctgaaagtccgtatgccggaggagtgtttttggtttctattcatttccctccggattatcctttcaagccaccaaaggtgtcttttaagactaaggtgtaccatccaaacatcaatagcaatggaagtatttgccttgatattttgaaagagcaatggagccctgctcttactatctctaaggttctgttgtcgatttgctcgctgttgactgacccaaacccagatgatcctcttgtgccggagatagctcacatgtacaagacagatagatccaagtacgagtcaactgcgagaagctggacacaaaagtacgcaatgggttga

SEQ ID NO: 17 - DNA, 486 bp, Arabidopsis thaliana
atgtcaagttctggtgctccttcacgcaagactctgagtaagatcgcgactaatcgtcttcagaaagagcttgtggaatggcagatgaatccacctactggtttcaaacacaaagtcactgataatctccaaagatggataattgaagttattggagctccaggaactctatatgccaacgatacttatcagcttcaagttgattttccagaacattatcctatggaatcgccacaagtgatttttcttcatccagctcctctgcatcctcacatttacagcaatgggcatatttgtctagatattttgtatgattcgtggtctccagccatgacggtgagttctatctgcattagcatcctctccatgctctcaagctcgactgaaaagcaacgaccaaccgataatgaccgatatgtgaagaattgtaagaacggaagatctccaaaggagacccgatggtggttccacgacgataaagtataa

SEQ ID NO: 18 - DNA, 486 bp, Arabidopsis thaliana
atgacaagctcttcatctccttcacgcaaggcgttaagcaagatcgcgtgtaataggttacaaaaggagttatcagagtggcaagtaaatccaccgactgggtttaagcacagagttactgataatcttcaaaaatgggtaattgaggtaacaggagcacctggtacgctttacgctaatgaaacttataatcttcaggttgagtttcctcaacattatcctatggaagctcctcaggtgatttttgttccaccagcaccgttacatcctcatatttacagcaatggacatatttgtttagatattctgtatgactcatggtctccagcaatgacagtgagttcagtctgcatcagcatcctctcaatgctatcaagttcccccgaaaagcaacgcccgactgataacgatcgatatgtgaagaactgcaagaacgggaggtctcctaaagagacaaggtggtggttccatgacgacaaggtttaa

SEQ ID NO: 19 - DNA, 756 bp, Arabidopsis thaliana
atggctagtaatgagaatctaccacccaatgttatcaagcagcttgccaaggagctgaagagtcttgacgaatcccctccagatggcatcaaggttgtggtaaatgacgaggatttctcacaaatatgtgctgatattgaaggaccagttggtactccttatgagaacggacttttccgtatgaagttggccttgtcacatgattttccacattcgcctcccaaaggctacttcatgacaaagattttccatcctaatgttgcttctaatggagagatatgtgttaacacgcttaaaaaagattggaaccctagtcttggattaagacatgttctctctgttgtaaggtgcttattaatcgagccatttccagaatctgcattaaatgaacaagctggcaagatgctacttgaaaactatgacgagtatgcgaggcatgctaggctttacacaggtatccatgctaaaccaaaacccaagttcaagacaggagcaatctcagagtcaacaaccgctctaaatgttggccagacgaacaacgagacgcctggtgctgctactgcaataccctcttcaatgacagacataaagagagtaacaacgagtgcccaagaccaacaacacgtagctaatgtcgttgtagcagcctctgcaagtgtggtcactacgacgcaaaaaagggaagcaggtttggccaaggttcaggcagataagaagaaggtagacgctagaaagaaaagcttgaagagactatga

SEQ ID NO: 20 - DNA, 2724 bp, Arabidopsis thaliana
atggaaatgtcccttactgactctgattgggatagctccagcgacagtggtagcagtgaacacgaagaagtcgagttttcttatggtggacgggcacagaacattttctcaaaccttgaagaaaccattggcaaaatcgatgagttcctgtcgttcgagaggggatttatgtatggtgacattgtgcggtccgccactgaaccatcaggacagagtggcagggttatcaacatagacatgtttgtcaatctcgaaagtactcatgggaagatcatgaaggaagttgataccaagaggcttcaaaagttgcgttctatttcactctctgattatgtgattaacggaccttgggttggaagggttgacaaaatagttgagcgtgtctctgtcacccttgatgatgggaccaactatgaggtccttgtagatggtcaagataaacttgtggccattcccccaaatttactcgaggattctcaatattcgtattacccagggcaaagagttcaggtaaagctggcccatgcccccagatcaactacatggttatgcgggacctggagaggaacccaggttatgggaactgtttgcactgtagaagcaggacttgtctacgtcgattgggttgcctccatcgtaatggagggtgatcggaatttaactgcacctcaagctttgcagaatcctgagagtttaactttgttaccttgtgtttctcatgcgagttggcagcttggtgactggtgtatactccctggctcttcccactgtgatatagcagagcggcaaactccaaatgtggctgcctacaatctcaatgaatgccataagacattccaaaaagggtttaacagaaatatgcagaactcaggtttggatgagctatttgttatcacaaagacaaagatgaaggttgctgttatgtggcaagatggtagctgcagtctgggagttgattcccaacagctgcttcctgttggtgctgttaatgctcatgatttttggcccgaacagtttgttgtggaaaaggaaacctgcaacagcaaaaaatggggagttgtgaaggctgtcaatgctaaggaacaaactgtgaaggtacaatggacaatacaggttgagaaagaagcaacaggttgtgttgatgaggtgatggaagaaattgtcagtgcatatgaactgcttgagcaccctgattttggattctgtttcagcgatgtggtggtcaagttacttccagaaggaaaatttgatccaaatgcagacacaatcgtcgctacagaggcgaaacacctacttacagagagtgactacagtggcgcatattttttgtcaagtattggtgttgttacaggttttaaaaatggttccgtgaaggtgaaatgggccaatggttctactagcaaggttgcaccatgtgaaatttggaaaatggaaaggtctgaatattccaactctagcactgtaagttcggaaggcagtgttcaagatctaagtcagaagattagtcaatcggatgaagcatcttcaaaccatcaggaaacgggtctggtgaagctctacagtgttggtgaaagttgcaacgagaacattccggaatgtagttcatttttccttccaaaagctgccattggatttatcacaaaccttgcatcaagcctttttggttatcagggttccacttcggttataagttcacattcacgttgcaatgattctgaagatcaaagtgactctgaggtccttgttcaagaaacagcagaatcatatgacaactctgaaacaaattcaggtgaagtggatatgaccaccacgatggtcaacatacctatagaaggaaaaggaattaacaagacactggattcaactcttctagagaacagcaggaaccaagtgagattcagacagtttgatatggttaatgactgctcggaccatcatttcctttcttcggataaagggttggctcagtcccaggtcacaaagagttgggtgaagaaagtccagcaagaatggagcaatttggaggcaaatcttccgaacacgatatatgtgcgtgtgtgtgaagaaaggatggaccttctgcgtgcagccctggttggtgcccctggaacgccatatcacgatgggcttttctttttcgacataatgcttccacctcaatatcctcatgagccaccaatggtacattatcattcaggtgggatgcgactgaatccgaacctgtatgagtcaggaagagtttgcttgagtctgctgaatacatggagtggctctggcactgaagtatggaacgcagggagctcctccatccttcaacttcttctttcgtttcaggctctggttctgaatgagaagccttacttcaatgaagctggctatgataagcagttgggccgagccgagggagagaaaaactcagtgagttacaatgagaatgcattcctcataacctgcaaatccatgatctcaatgctccgtaagcctccaaagcattttgagatgcttgtgaaggaccattttacgcaccgggcccagcatgttctggctgcgtgcaaggcttatatggaaggcgtccctgtaggatcatcagctaacctgcaggggaactcaaccacaaattccaccggtttcaagatcatgctctccaaactctacccaaaacttcttgaagcattctcagagattggagttgattgcgtccaagagattggaccagaatcataa

SEQ ID NO: 21 - DNA, 579 bp, Arabidopsis thaliana
atgatagatttcagtcgaatccagaaagagcttcaggactgcgagagaaaccaggattcttcgggtatccgggtctgccccaaatccgataacctcacccgactcaccggaaccattcctggtccaatcggtactccttacgaaggcggtacatttcagatcgatatcactatgccagatgggtatccatttgagcctccaaagatgcagttttcaactaaagtttggcatccgaatattagtagccaaagcggtgcaatatgcttggatatcttgaaagaccagtggagtccagctcttactctgaagacagctcttgtttcaattcaggctttactctctgcacctgagcccaaagaccctcaagatgctgttgtagctgaacagtacatgaagaactatcaagtgtttgtatcaacagctcgttactggactgaaactttcgccaagaaatcttctctagaggaaaaagtcaagagacttgtggagatgggttttggagacgctcaagttaggagtgcaattgaatcaagtggtggtgatgagaatctggctctggaaaagctctgttctgcttag

SEQ ID NO: 22 - DNA, 447 bp, Arabidopsis thaliana
atggcttcgaaaaggatcttgaaagagctcaaggatctccagaaggatcctccaacttcctgcagtgctggcccagttgctgaagacatgtttcattggcaagctacaataatgggtccatctgatagtccttattcaggcggtgtctttctcgtaaccattcacttccctccggattatcctttcaaaccaccaaaggttgcattcaggacaaaagtgttccaccctaatgtcaacagcaatggaagcatttgccttgacattttgaaagaacaatggagtcctgcactcaccatatcgaaggttttgctttcgatatgttcattgttaacggacccaaacccagatgatccattggttccagagattgctcacatgtacaaaaccgatagagcaaagtatgagtctactgcgagaagctggactcagaaatatgcaatgggatga

SEQ ID NO: 23 - DNA, 447 bp, Arabidopsis thaliana
atggcaacgagacggatattgaaagaattgaaggagttgcaaagagaccctcctgtatcatgcagtgcaggtccaacaggagaagatatgttccactggcaagctactataatgggtccgaatgaaagtccttactccggaggtgttttccttgtcaatattcatttccctcctgattatccttttaaacctcccaaggttgtattcagaaccaaagtgtttcacccaaacatcaacagtaatggaaacatatgtttggacattctcaaagaccaatggagccctgcccttaccatttctaaggtgcttctctctatatgctctcttctaacagatccaaaccctgacgaccctctggtgccagagatagctcacatatacaaaactgacaagaccaaatatgaagccatggctcgaagctggacccagaagtatgccttgttttaa

SEQ ID NO: 24 - DNA, 465 bp, Arabidopsis thaliana
atgtttaagaaaatggataaaaaagcagcgcagagaattgcgatggaatacagagctatgatctcgaaagaatctttgttcagtattggtcaaaactcgaacaatatatacgaatggactgcagtgatccggggtccagatggcactccctatgaaggtggcatgtttaatctcagtattaagtttcctacggattatccttttaaaccacccaagtttacgtttaaaactccgatttaccatccaaatatcaatgatgaaggatcgatttgcatgaacattcttaaagacaaatggactcctgctcttatggttgaaaaggtgcttctgtcaatacttttactattggaaaaaccaaacccagatgatcctcttgtacctgaaattggacagctcttcaagaataacagattccaatttgaccagagagctcgagaattcactgctcgacatgctaataattaa

SEQ ID NO: 25 - DNA, 660 bp, Arabidopsis thaliana
atggcagaaaaagcttgtataaagcgtcttcaaaaagaatacagagcgctttgcaaggaaccagtctcgcatgttgttgctcgtccttccccaaatgacattcttgaatggcattatgtgttggaaggcagtgagggaacgccttttgcaggtggattttactatggaaagatcaagttccctccagaatatccttacaagccacctggaatcacaatgaccacaccaaatggtcgatttgtgacgcaaaagaaaatctgcttgtctatgagtgactttcatccagaaagctggaatccaatgtggtcagtgtcaagcatacttacaggacttctctcatttatgatggataacagccccacaacgggaagtgtaaacactagtgtagccgagaaacaacggttggcgaagtcatctcttgctttcaattgtaaaagtgtaacattccggaaactatttcccgagtatgtcgagaagtacagccagcaacaagtagctgaagaagaagcagccactcaacagacaacaacatctgagaatcaggattttcctcagaaggacaatgcgaaagtcgaatcagagaaaagcgttggtcttaaaaaggaaagtattcaagaagtgggattgaaagagaggagaaggaacaagaaagaagcataa

SEQ ID NO: 26 - DNA, 642 bp, Arabidopsis thaliana
atggcagaaaaggcctgtataaaacgtcttcaaaaggaatatagagcactttgcaaggaaccagtctcccatgttgttgctcgtccttccccaaatgacattctcgagtggcattatgtgctggaaggcagtgagggaacaccttttgcaggtggattttactatggaaagatcaagtttcctcccgagtatccttacaagccaccagggattacaatgactacaccaaatggtcgattcatgacacagaagaaaatttgtttatctatgagtgattttcatccagaaagctggaatccgatgtggtctgtatcaagcatacttacaggacttctctcatttatgatggataccagtccgacaactggaagtgttaacactactgtaattgagaaacaacggttggctaagtcatctctcgctttcaattgtaaaaccccagcatttcgaaagctatttccagagtatgtagagaagtacaaccagcagcaactagctgagcaagctaccacacaactgacaacaccagagtctcctcaaaagagcgataccaaagtcgagtcagagaaaaccatcgatccaacaaagggagattcagaaggtggcttgaaggagaggaaaaagaacaataaacagggatga

SEQ ID NO: 27 - DNA, 462 bp, Arabidopsis thaliana
atggccaatagtaatcttccccgacgaatcatcaaggaaactcaacgtctgcttagtgaacctgctcctgggataagtgcgtctccatcagaggaaaatatgagatatttcaatgttatgattcttggtcctactcaatctccttatgaaggaggagttttcaagttggagctctttttgcctgaagaatatcctatggcagctcccaaggttaggtttctcaccaagatttaccatcctaacatcgacaagcttggaagaatctgccttgacatactgaaagacaagtggagtcctgcactacaaatacgaactgtgctcttgagtatccaagctcttttaagtgcaccaaacccagatgatcctctgtctgagaacatagctaagcattggaagagcaatgaggcagaagctgtggaaacagctaaggaatggactcgtctttacgcaagtggtgcttaa

SEQ ID NO: 28 - DNA, 36 bp, Arabidopsis thaliana
gacaagggtaccatggctggtggagctatcactccc

SEQ ID NO: 29 - DNA, 34 bp, Arabidopsis thaliana
gacaaggcggccgcgagtgaacctaattttcggg

SEQ ID NO: 30 - DNA, 29 bp, Arabidopsis thaliana
agaggactttcttgctccaatctctctgg

SEQ ID NO: 31 - DNA, 29 bp, Arabidopsis thaliana
ccagagagattggagcaagaaagtcctct

SEQ ID NO: 32 - DNA, 35 bp, Arabidopsis thaliana
gacaagggtaccgagattgtttgctaagaaaatgg

SEQ ID NO: 33 - DNA, 34 bp, Arabidopsis thaliana
gacaaggcggccgctacgccgtctcacacggcgg

SEQ ID NO: 34 - DNA, 35 bp, Arabidopsis thaliana
gacaagggtaccatggccggaggaatcgtctcacc

SEQ ID NO: 35 - DNA, 34 bp, Arabidopsis thaliana
gacaaggcggccgcttggcatgctttacgaagaa

SEQ ID NO: 36 - DNA, 29 bp, Arabidopsis thaliana
ggttgattttcttgctccggtgtcgcttg

SEQ ID NO: 37 - DNA, 29 bp, Arabidopsis thaliana
caagcgacaccggagcaagaaaatcaacc

SEQ ID NO: 38 - DNA, 36 bp, Arabidopsis thaliana
gacaagggtaccctctaccctccagtttctagctcc

SEQ ID NO: 39 - DNA, 34 bp, Arabidopsis thaliana
gacaaggcggccgctacgccgtcgccgatcaacc

SEQ ID NO: 40 - DNA, 35 bp, Arabidopsis thaliana
gacaagggtaccatgactgattaccggctacaacc

SEQ ID NO: 41 - DNA, 34 bp, Arabidopsis thaliana
gacaaggcggccgcaccgatttttgaaatcaaac

SEQ ID NO: 42 - DNA, 33 bp, Arabidopsis thaliana
gacaagggtaccctagtggcgtcacccccaaag

SEQ ID NO: 43 - DNA, 32 bp, Arabidopsis thaliana
gacaagctcgagtccataaaccggtgaccggt

SEQ ID NO: 44 - DNA, 21 bp, Arabidopsis thaliana
acctcaccatggagcaaaagc

SEQ ID NO: 45 - DNA, 23 bp, Arabidopsis thaliana
agattcatcgttggttgtagccg

SEQ ID NO: 46 - DNA, 21 bp, Arabidopsis thaliana
agaaggattgttccgatctcg

SEQ ID NO: 47 - DNA, 24 bp, Arabidopsis thaliana
acacatcaaagtttagagagctcc

SEQ ID NO: 48 - DNA, 20 bp, Arabidopsis thaliana
tggtggagctatcactcccg

SEQ ID NO: 49 - DNA, 20 bp, Arabidopsis thaliana
tggtggagctatcactcccg

SEQ ID NO: 50 - DNA, 1917 bp, Zea mays
  CDS (1)..(1914)
atggcggcggcggcggcggcggagatcgtgcgggagatcgcagcggtgggcgccgccgacctggccgccgccgccgatccgctgcgcgcggactgcctccgcctcgcgcggaaggtgtccctgctctcgcacctcgtggccgaggtcgccgacgccggggccggggccggggcgggcgatgcggcggcggaggcgtggctgcgcgacctcgtcagggcgctcgaggctgcccggaggtttgtcgcgctcggacggacgccggccgctgcgggggcgtcagatcaggatgccatctgcaagaatactggtcttcagttcaagtatgtgacctggcagttgcaagctgctctggcaaacctgccacatagctgttttgagatatcagacgaagttcaagaagaggttgacttagtgcgagctcagcttagaagagaaatggaaaagaatggaggtcttgatgtaaccgtatttatgaaagttcatgatatcttagctcaaattgacaatgctgcaccacaatctcaacagccgcatcaacaaccagagcagccacagatggagaaattcagtaatgatgacctggagttgcgaaatgtcgtattgctagtttctgaaataagcgggttatccaaacctgacatgtcaaaaatagtatcagatctaattcaaggacttgaaaatgttgaaactcctgagtctccgaaaccagacaacgttgatcgccaaccaccaagtgatgaaacaaaaggctcatctgaggaagttaagaagtcggactctgtagctattcctgaagatttccgttgcccaatatctctcgagctgatgagagatccagtaattgtttcaacgggacagacctatgagcgcgctttcattcagaggtggattgactgtggaaaccgaacctgcccaaaaactcagcagaagctccagaacctttcgttgaccccaaactacgtgcttagaagcttaatactgcagtggtgcgaggaaaaggggatggaaccacctagcaggtctaaaagcgacggttctcctcttgaggttgctggcaacagattagcaattgaagcgttagtccgcaacctctcgagcagttcattagatgatcggaaatctgctgcggctgaaataagatccttggccaagaaaagcacagacaaccgtatacttctagcggaatctagcgctataccggctctggtgaaactcctgtcgtccaaggacccgaaaacccaagagcacgcagtgacagctctcctgaacctctccatatacgatcagaacaaggagctggtagtggttgccggtgccatcgtcccaatcacacaggtgctgaggacgggtagcatggaagcaagagagaacgcagccgcggctatcttcagcctgtcactgatggacgacaacaagatcatgatagggagcaccccaggggcaatcgaggcgctggtggagctgctgcagagcggtagctcgaggggcaagaaggacgcggcgacagcgctgttcaacctgtgcatataccaggcgaacaaggtgagggccgtccgagcggggattctggtgccgctgatccggatgctgcaggactcgtccaggagcggagccgtcgacgaggcgctgacgatcctgtcggtcctcgcgagccaccacgagtgcaagaccgccatatccaaggcccacgccatacctttcctgatcgacttgctgcggtcaggccaggcccgcaacagggagaacgctgccgccatcatactcgcgctgtgcaagagagacgccgagaacctcgcctgcgtagggaggctgggcgcccagatcccgctcgccgagctggcgaagaccggcacggaccgagccaagcgcaaggcaacctcgctcctggagcacctcagcaagctgcaggtgctctag

SEQ ID NO: 51 - protein, 638 aa, Zea mays
MAAAAAAEIVREIAAVGAADLAAAADPLRADCLRLARKVSLLSHLVAEVADAGAGAGAGDAAAEAWLRDLVRALEAARRFVALGRTPAAAGASDQDAICKNTGLQFKYVTWQLQAALANLPHSCFEISDEVQEEVDLVRAQLRREMEKNGGLDVTVFMKVHDILAQIDNAAPQSQQPHQQPEQPQMEKFSNDDLELRNVVLLVSEISGLSKPDMSKIVSDLIQGLENVETPESPKPDNVDRQPPSDETKGSSEEVKKSDSVAIPEDFRCPISLELMRDPVIVSTGQTYERAFIQRWIDCGNRTCPKTQQKLQNLSLTPNYVLRSLILQWCEEKGMEPPSRSKSDGSPLEVAGNRLAIEALVRNLSSSSLDDRKSAAAEIRSLAKKSTDNRILLAESSAIPALVKLLSSKDPKTQEHAVTALLNLSIYDQNKELVVVAGAIVPITQVLRTGSMEARENAAAAIFSLSLMDDNKIMIGSTPGAIEALVELLQSGSSRGKKDAATALFNLCIYQANKVRAVRAGILVPLIRMLQDSSRSGAVDEALTILSVLASHHECKTAISKAHAIPFLIDLLRSGQARNRENAAAIILALCKRDAENLACVGRLGAQIPLAELAKTGTDRAKRKATSLLEHLSKLQVL

SEQ ID NO: 52 - DNA, 300 bp, Zea mays
gtcagggcgctcgaggctgcccggaggtttgtcgcgctcggacggacgccggccgctgcgggggcgtcagatcaggatgccatctgcaagaatactggtcttcagttcaagtatgtgacctggcagttgcaagctgctctggcaaacctgccacatagctgttttgagatatcagacgaagttcaagaagaggttgacttagtgcgagctcagcttagaagagaaatggaaaagaatggaggtcttgatgtaaccgtatttatgaaagttcatgatatcttagctcaaattgacaatgct

SEQ ID NO: 53 - DNA, 459 bp, Sorghum bicolor
aactatctatactgtaataatgttgtatagccgccggatagctagctagtttagtcattcagcggcgatgggtaataataaagtgtcatccatccatcaccatgggtggcaacgtgagcaatgacctgattgaacaaattgaaatgaaaagaagaaatatgttatatgtcaacgagatttcctcataatgccactgacaacgtgtgtccaagaaatgtatcagtgatacgtatattcacaatttttttatgacttatactcacaatttgtttttttactacttatactcacaatttgttgtgggtaccataacaatttcgatcgaatatatatcagaaagttgacgaaagtaagctcactcaaaaagttaaatgggctgcggaagctgcgtcaggcccaagttttggctattctatccggtatccacgattttgatggctgagggacatatgttcgctt

(19) United States
(12) Patent Application Publication
(10) Pub. No.: US 2017/0306346 A1
(43) Pub. Date: Oct. 26, 2017

(54) IMPROVED AGRONOMIC CHARACTERISTICS UNDER WATER LIMITING CONDITIONS FOR PLANTS EXPRESSING PUB10 POLYPEPTIDES

(71) Applicant: THE ROCKEFELLER UNIVERSITY, New York, NY (US)

(72) Inventors: Nam-Hai CHUA, New York, NY (US); Choonkyun JUNG, New York, NY (US); Pingzhi ZHAO, Nanjing (CN)

(73) Assignee: THE ROCKEFELLER UNIVERSITY, New York, NY (US)

(21) Appl. No.: 15/520,081

(22) PCT Filed: Nov. 10, 2015
(86) PCT No.: PCT/US15/59933

§ 371 Date: Apr. 19, 2017

Related U.S. Application Data
(60) Provisional application No. 62/078,692, filed on Nov. 12, 2014.

(51) Int. Cl. C12N 15/82; C07K 14/415; C12N 15/00; A01H 1/00

(52) U.S. Cl. CPC C12N 15/8273; C07K 14/415; C12N 15/8218; A01H 1/00; C12N 15/00

(57) ABSTRACT

The present invention provides methods and compositions for modulating drought tolerance and/or other agronomic traits in plants. This invention relates to compositions and methods for down-regulating the level and/or activity of PUB10 in plants for creation of plants with improved abiotic stress tolerance, preferably improved drought tolerance. Thus, in one aspect, the present invention provides an isolated nucleic acid comprising a polynucleotide sequence for use in a recombinant DNA construct or a suppression DNA construct for modulating PUB10 expression.

### CROSS-REFERENCE TO RELATED APPLICATIONS

The present application is related to and claims priority to U.S. provisional patent application Ser. No. 62/078,692 filed on 12 Nov. 2014. Each application is incorporated herein by reference in their entirety.

### SEQUENCE SUBMISSION

The present application is being filed along with a Sequence Listing in electronic format. The Sequence Listing is entitled 2312135PCTSequenceListing.txt, created on 28 Oct. 2015 and is 85 kb in size. The information in the electronic format of the Sequence Listing is incorporated herein by reference in their entirety.

### BACKGROUND OF THE INVENTION

The present invention relates to the field of plant breeding and genetics, and in particular relates to recombinant DNA constructs useful in plants for conferring tolerance to drought.

The publications and other materials used herein to illuminate the background of the invention or provide additional details respecting the practice are incorporated by reference. Full citations of references referred to in the text by author and publication year are set forth in the Bibliography.

Abiotic stress is the primary cause of crop loss worldwide, causing average yield losses of more than 50% for major crops. Among the various abiotic stresses, drought is the major factor that limits crop productivity worldwide. Exposure of plants to a water-limiting environment during various developmental stages appears to activate various physiological and developmental changes. Understanding of the basic biochemical and molecular mechanism for drought stress perception, transduction and tolerance is a major challenge in biology. Reviews on the molecular mechanisms of abiotic stress responses and the genetic regulatory networks of drought stress tolerance have been published (Valliyodan and Nguyen, 2006).

Regulated proteolysis plays important roles in plant signaling pathways in at least two important steps. In several pathways, signaling is restrained by repressors (auxin, JA, GA) but upon signal perception these repressors are destroyed by ubiquitin-mediated proteolysis to initiate signaling. In other pathways, ubiquitin-mediated proteolysis is used to degrade positive components, e.g. receptors or transcription factors, to down regulate and terminate signaling (FLS signaling, etc.).

Like other eukaryotes, ubiquitin-dependent protein degradation in plants is carried out by 3 enzymes acting in sequential steps: E1 ubiquitin-activating enzyme, E2 ubiquitin-conjugating enzyme and E3 ubiquitin ligase, with substrate specificity being conferred by the E3 ligase. Arabidopsis thaliana genes coding for more than 1,300 E3 ligases including proteins with RING motif, HECT domain, F-box proteins and U-box have been annotated (Vierstra, 2009). Among these various E3 categories a large number of RING-motif proteins and SCF complexes have been investigated with respect to their molecular functions in plant growth and development. Compared to RING proteins and SCF complexes, much less is known about the U-box proteins, which are also known as PUBs (plant U-box) (Yee and Goring, 2009).

The Arabidopsis genome encodes at least 64 PUBs and approximately 40% of them have been shown to have E3 activities when associated with specific UBCs. (Mudgil et al., 2004; Wiborg et al., 2008) Whereas the biochemical properties and NMR structure of a PUB protein have been determined (Andersen et al., 2004; Wiborg et al., 2008) the biological function of only a limited number of PUBs is known (Yee and Goring, 2009). For example, PUB9, 18 & 19 have been linked to ABA responses (Samuel et al., 2008; Bengler &n Hoth, 2011; Seo et al., 2012), PUB12, 13, 17, 22, 23 and 24 all play a role in various steps of the innate immunity pathway (Yang et al., 2006; Cho et al., 2008; Trujilio et al., 2008; Lu et al., 2011) and PUB22 and 23 are also associated with drought responses as their over-expression plants displayed hypersensitivity (Cho et al., 2008). In addition to Arabidopsis, PUBs of other plants have been also been implicated in biotic and abiotic stresses (for a review, see Yee and Goring, 2009)

It is desired to characterize the biological function of previously uncharacterized Arabidopsis PUB10. It is also desired to identify and characterize homologs of Arabidopsis PUB10 in other plant species.

### SUMMARY OF THE INVENTION

The present invention provides methods and compositions for modulating drought tolerance and/or other agronomic traits in plants. This invention relates to compositions and methods for down-regulating the level and/or activity of PUB10 in plants for creation of plants with improved abiotic stress tolerance, preferably improved drought tolerance. Thus, in one aspect, the present invention provides an isolated nucleic acid comprising a polynucleotide sequence for use in a recombinant DNA construct or a suppression DNA construct for modulating PUB10 expression.

In one embodiment, the present invention provides a plant comprising in its genome a suppression DNA construct comprising at least one regulatory element operably linked to a region derived from all or part of a sense strand or antisense strand of a target gene of interest, said region having a nucleic acid sequence of at least 90% sequence identity, based on the Clustal V or Clustal W method of alignment, when compared to said all or part of a sense strand or antisense strand from which said region is derived, and wherein said target gene of interest encodes a PUIB10 polypeptide, and wherein said plant exhibits increased drought tolerance when compared to a control plant not comprising said suppression DNA construct. The plant may further exhibit an alteration of at least one agronomic characteristic when compared to the control plant.

In another embodiment, the present invention provides a plant comprising in its genome a suppression DNA construct comprising at least one regulatory element operably linked to all or part of (a) a nucleic acid sequence of at least 90% sequence identity, based on the Clustal V or Clustal W method of alignment, when compared to SEQ ID NO:1 or 50, or (b) a full complement of the nucleic acid sequence of (a), and wherein said plant exhibits increased drought tolerance when compared to a control plant not comprising said suppression DNA construct. The plant may further exhibit an alteration of at least one agronomic characteristic when compared to the control plant.

In a further embodiment, the present invention provides a plant comprising in its genome a recombinant DNA construct comprising a polynucleotide operably linked to at least one regulatory sequence, wherein said polynucleotide encodes all or part of a polypeptide having an amino acid sequence of at least 90% sequence identity, based on the Clustal V or Clustal W method of alignment, when compared to SEQ ID NO:2 or 51, and wherein said plant exhibits increased drought tolerance when compared to a control plant not comprising said recombinant DNA construct. The plant may further exhibit an alteration of at least one agronomic characteristic when compared to the control plant.

In an additional embodiment, the present invention includes any of the plants of the present invention wherein the plant is selected from the group consisting of: Arabidopsis, maize, soybean, sunflower, sorghum, canola, wheat, alfalfa, cotton, rice, barley, millet, sugar cane and switchgrass.

In another embodiment, the present invention includes seed of any of the plants of the present invention, wherein said seed comprises in its genome a suppression DNA construct or recombinant DNA construct described herein and wherein a plant produced from said seed exhibits drought tolerance when compared to a control plant not comprising said suppression DNA construct or recombinant DNA construct.

In a further embodiment, the present invention provides a method of increasing drought tolerance in a plant, comprising: (a) introducing into a regenerable plant cell a suppression DNA construct or recombinant DNA construct described herein and (b) regenerating a transgenic plant from the regenerable plant cell after step (a), wherein the transgenic plant comprises in its genome the suppression DNA construct or recombinant DA construct and exhibits increased drought tolerance when compared to a control plant not comprising the suppression DNA construct or recombinant DNA construct. The method may further comprise (c) obtaining a progeny plant derived from the transgenic plant, wherein said progeny plant comprises in its genome the suppression DNA construct or recombinant DNA construct and exhibits increased drought tolerance when compared to a control plant not comprising the suppression DNA construct or recombinant DNA construct.

In an additional embodiment, the present invention provides a method of selecting for (or identifying) increased drought tolerance in a plant, comprising (a) obtaining a transgenic plant, wherein the transgenic plant comprises in its genome a suppression DNA construct or recombinant DNA construct described herein; (b) obtaining a progeny plant derived from the transgenic plant, wherein the progeny plant comprises in its genome the suppression DNA construct or recombinant DNA construct; and (c) selecting (or identifying) the progeny plant with increased drought tolerance compared to a control plant not comprising the suppression DNA construct or recombinant DNA construct.

In another embodiment, the present invention provides a method of selecting for (or identifying) an alteration of at least one agronomic characteristic in a plant, comprising: (a) obtaining a transgenic plant, wherein the transgenic plant comprises in its genome a suppression DNA construct or recombinant DNA construct described herein, wherein the transgenic plant comprises in its genome the suppression DNA construct or recombinant DNA construct; (b) growing the transgenic plant of part (a) under conditions wherein the polynucleotide is expressed in the suppression DNA construct or recombinant DNA construct; and (c) selecting (or identifying) the transgenic plant of part (b) that exhibits an alteration of at least one agronomic characteristic when compared to a control plant not comprising the suppression DNA construct or recombinant DNA construct. Optionally, said selecting (or identifying) step (c) comprises determining whether the transgenic plant exhibits an alteration of at least one agronomic characteristic when compared, under water limiting conditions, to a control plant not comprising the suppression DNA construct or recombinant DNA construct.

In a further embodiment, the present invention includes any of the methods of the present invention wherein the plant is selected from the group consisting of: Arabidopsis, maize, soybean, sunflower, sorghum, canola, wheat, alfalfa, cotton, rice, barley, millet, sugar cane and switchgrass.

### BRIEF DESCRIPTION OF THE FIGURES

FIGS. 1A-1C show that PUB10 and PUB11 interact with MYC2 via its armadillo repeats (ARM). FIG. 1A: Full-length cDNA fragments of MYC2 (SEQ ID NO:5), PUB10 (SEQ ID NO:1) and PUB11 (SEQ ID NO:3) were fused to sequences encoding GAL4 activation domain (AD) and GAL4 DNA-binding domain (BD) in pGAD424 and pGBT9, respectively. The two vectors were transformed into the yeast strain AH109. Transformants were plated onto minimal medium -Leu/-Trp, -Leu/-Trp/-His or -Leu/-Trp/-His/3-AT. Protein interactions were shown by colony growth. C1: AD+BD; C2: AD-MYC2+BD; C3: AD+BD-PUB10; C4: AD+BD-PUB11; 3-AT: 3-Amino-1,2,4-triazole. FIG. 1B: Schematic diagram of full-length PUB10 and its deletion derivatives used to test for PUB10 and MYC2 interaction (upper panel). MBP, MBP-PUB10 or its deletion derivatives were used as baits. GST-MYC2 was used as a prey. Cys249 of PUB10 U-box was changed to Ala (lower panel). Full length PUB10 is SEQ ID NO:2; PUB10-(UND) is 1-240 aa of SEQ ID NO:2; PUB10-(U-box) is 240-320 aa of SEQ ID NO:2; PUB10-(ARM) is 320-628 aa of SEQ ID NO:2. FIG. 1C: Wild type PUB10 and mutant PUB10 (C249A) form a dimer and interact with MYC2. GST: GST-PUB10 or GST-PUB10 (C249A) was used as baits. MBP, MBP-PUB10 or MBP-PUB10 (C249A) was used as preys. For (FIG. 1C), two micrograms of prey proteins were pulled-down with the indicated bait proteins (2 ug each) and detected by anti-GST and anti-MBP antibodies, respectively.

FIGS. 2A and 2B show that PUB10 is a ubiquitin E3 ligase and ubiquitinates MYC2 in vitro. FIG. 2B: PUB10 is a ubiquitin E3 ligase and PUB10 E3 activity is dependent on the integrity of its U-box motif. Epitope-tagged recombinant PUB10, PUB10 (C249A), and AtUBC8 proteins were purified from E. coli extracts. MBP-PUB10-myc and MBP-PUB10 (C249A)-myc were assayed for E3 activity in the presence or absence of human E1 (UBE1), Arabidopsis E2 (AtUBC8) and 6× His-ubiquitin (Ub). FIG. 2B: MYC2 is a substrate of PUB10 E3 ligase. MBP-PUB10-myc E3 activity was assayed in the presence or absence of E1, E2, Ub and GST-MYC2-HA. MBP-PUB10 (C249A)-myc mutant protein has no E3 activity for GST-MYC-HA. For FIGS. 2A and 2B, polyubiquinated PUB10 and MYC2 were detected by anti-myc and anti-HA antibodies, respectively.

FIGS. 3A-3D show the interaction of PUB10 and MYC2 in tobacco and Arabidopsis. FIG. 3A: Colocalization of PUB10-YFP and MYC2-CFP in the nucleus. Fluorescent fusion genes (PUB10-YFP and MYC2-CFP) were transiently expressed in N. benthaminana leaves infiltrated with agrobacterial cultures. FIG. 3B: Bimolecular fluorescence analysis of the interaction between PUB10 (C249A) and MYC2 in tobacco. Reconstituted YFP signals were detected in the nucleus of tobacco leaf cells when MYC2-nYFP was coexpressed with cYFP-PUB10 (C249A). Coexpression of nYFP or cYFP with the corresponding cYFP-PUB10 (C249A) or MYC2-nYFP constructs was used as an additional control. FIGS. 3C and 3D: Coimmunoprecipitation of PUB10 with MYC2 in Arabidopsis. Two-week-old double transgenic Arabidopsis seedlings carrying 35S:myc-MYC2/XVE:HA-PUB10 or 35S:myc-MYC2/XVE:HA-PUB10 (C249A) were treated for 16 h with 50 uM MG132 in the absence or presence of 25 uM β-estradiol. Extracts were immunoprecipitated with anti-myc or anti-MBP antibodies. Input proteins and the immunoprecipitates were analyzed by Western blots using anti-HA and anti-myc antibodies. Input refers to the starting protein amount in extracts used for IP reactions.

FIGS. 4A and 4B shows histochemical localization of GUS activity in transgenic plants carrying PUB10 and MYC2 promoter-GUS. FIG. 4A: An approximately 2.2-kb fragment of the PUB10 promoter was fused to the GUS gene and transformed into Arabidopsis. Histochemical assays for GUS activity in transgenic plants were performed as described by Jefferson et al. (1987). GUS expression was detected in one-week-old whole seedling (panel a), rosette leaves (panel b,c), petiole (panel d), lateral root (panel e), root tip (panel f), inflorescence (panel g), and silique (panel h). FIG. 4B: An approximately 3.0-kb fragment of the MYC2 promoter was fused to the GUS gene and transformed into Arabidopsis. GUS expression was detected in one-week-old whole seedling (panel a, c), cotyledon (panel b), hypocotyl (panel d), roots (panels e and f), inflorescence (panel g), and silique (panel h).

FIGS. 5A-5D show that PUB10 and MYC2 are targeted by PUB10 for degradation by 26S proteasomes. FIG. 5A: Seedlings of 35S:myc-PUB10, 35S:PUB10 (C249A)-myc and 35S:MYC2-GFP were incubated in liquid MS medium with or without 50 uM MG132 for 16 h. Protein levels were detected by anti-myc and anti-GFP antibodies. FIG. 5B: PUB10 expression is regulated post-translationally by self-ubiquitination. Transgenic seedlings of 35S:myc-PUB10 and 35S:PUB10 (C249A)-myc were incubated in liquid MS medium with 50 μM MG132 for 16 h, washed five times, and then transferred to liquid MS medium with 1 mM cycloheximide (CHX). Proteins were extracted at the indicated times and detected by anti-myc antibody. A cross-reaction band (arrow) is shown as a loading control. FIGS. 5C and 5D: Double transgenic Arabidopsis seedlings carrying 35S:myc-MYC2/XVE:HA-PUB10 or 35S:myc-MYC2/XVE:HA-PUB10 (C249A) were treated with β-estradiol alone, MG132 alone or β-estradiol plus MG132 for 16 h. Proteins were extracted at the indicated times and detected by anti-HA and anti-myc antibodies. Tubulin levels as detected by anti-tubulin antibody were used as loading controls. MYC2 transcript levels in each treatment were measured by real time RT-PCR.

FIGS. 6A-6D show that PUB10 is a negative regulator of MYC2 in ABA responses. FIGS. 6A-6C: The percentage of seeds showing radical emergence was scored 4 d post-stratification. Germination of the seeds was monitored from 0 to 7 d on MS medium containing different concentrations of ABA. Standard error bars represent three independent biological experiments. Three graphs (FIG. 6A, FIG. 6B, and FIG. 6C) share the same symbols: Col-0 (filled circle), myc2-1 (empty diamond), 35S:MYC2 (filled diamond), pub10-1(empty triangle), 35S:PUB10 (empty circle) and 35S:mPUB10 (filled triangle). FIG. 6B: Five-day-old seedlings germinated on MS medium were transferred to media containing 5 uM ABA, and the length of the primary root was measured 7d later.

FIGS. 7A and 7B show that PUB11 interacts with MYC2 via its armadillo repeats (ARM). FIG. 7A: Schematic diagram of full-length PUB11 and its deletion derivatives used to test for PUB11 and MYC2 interaction. FIG. 7B: MBP, MBP-PUB11 or its deletion derivatives were used as baits. GST-MYC2 was used as a prey. Cys247 of PUB11 U-box was changed to Ala. Two micrograms of prey proteins were pulled-down with the indicated bait proteins (2 ug each) and detected by anti-GST antibody. Full length PUB11 is SEQ ID NO:4; PUB11-(UND) is 1-235 aa of SEQ ID NO:4; PUB11-(U-box) is 235-320 aa of SEQ ID NO:4; PUB11-(ARM) is 320-612 aa of SEQ ID NO:4.

FIG. 8 shows that PUB10 and PUB11 interact with specific UBCs. Full-length cDNA fragments of PUB10, PUB11 and 35 UBCs were fused to sequences encoding GAL4 activation domain (AD) and GAL4 DNA-binding domain (BD) in pGAD424 and pGBT9, respectively. The two vectors were transformed into the yeast strain AH109. Transformants were plated onto minimal medium -Leu/-Trp, -Leu/-Trp/-His or -Leu/-Trp/-His/3-AT. Protein interactions were shown by colony growth. C1;AD-PUB10 or PUB11+BD, C2; AD+BD. 3-AT; 3-Amino-1,2,4-triazole.

FIGS. 9A and 9B show that PUB10 and PUB11 interact with specific UBCs in vitro. For FIGS. 9A and 9B, GST, GST-AtUBC2, GST-AtUBC8, GST-AtUBC31, and GST-AtUBC36 were used as preys. MBP-PUB10 and MBP-PUB11 were used as baits. Two micrograms of prey proteins were pulled-down with the indicated bait proteins (2 ug each) and detected by anti-MBP antibody.

FIGS. 10A and 10B show that specific UBCs support self-ubiquitination of PUB10 and PUB11 in vitro. FIG. 10A: Recombinant MBP-PUB10-myc, MBP-PUB10 (C249A)-myc, His-AtUBC2, His-AtUBC8, His-AtUBC16, His-AtUBC31, and His-AtUBC36 proteins were purified from E. coli extracts. MBP-PUB10-myc and MBP-PUB10 (C249A)-myc were assayed for E3 activity in the presence of human E1 (UBE1), human E2 (UbcH5b), Arabidopsis UBCs and 6× His-ubiquitin (Ub). FIG. 10B: Recombinant MBP-PUB11-myc, MBP-PUB11 (C247A)-myc, His-AtUBC2, His-AtUBC8, His-AtUBC10, His-AtUBC16, His-AtUBC31, and His-AtUBC36 proteins were purified from E. coli extracts. MBP-PUB11-myc and MBP-PUB11 (C247A)-myc were assayed for E3 activity in the presence of human E1 (UBE1), human E2 (UbcH5b), Arabidopsis UBCs and 6× His-ubiquitin (Ub). For (A) and (B), polyubiquitinated PUB10 and PUB11 were detected by anti-myc antibody.

FIGS. 11A and 11B show that PUB11 is a ubiquitin E3 ligase and ubiquitinates MYC2 in vitro. FIG. 11A: PUB11 is a ubiquitin E3 ligase and PUB11 E3 activity is dependent on the integrity of its U-box motif. Epitope-tagged recombinant PUB11, PUB11 (C247A), and AtUBC8 proteins were purified from E. coli extracts. MBP-PUB11-myc and MBP-PUB11 (C247A)-myc were assayed for E3 activity in the presence or absence of human E1 (UBE1), Arabidopsis E2 (AtUBC8) and 6× His-ubiquitin (Ub). FIG. 11B: MYC2 is a substrate of PUB11 E3 ligase. MBP-PUB11-myc E3 activity was assayed in the presence or absence of E1, E2, Ub and GST-MYC2-HA. MBP-PUB11 (C247A)-myc mutant protein has no E3 activity for GST-MYC-HA. For FIGS. 11A and 11B, polyubiquinated PUB11 and MYC2 were detected by anti-myc and anti-HA antibodies, respectively.

FIG. 12 shows that PUB11 protein is degraded by 26 S proteasomes. Seedlings of 35S:myc-PUB11, 35S:myc-PUB11 (C247A) were incubated in liquid MS medium with or without 50 uM MG132 for 16 h. Protein levels were detected by anti-myc antibody.

FIGS. 13A-13C show that PUB10 is a negative regulator in salt and osmotic responses. For FIGS. 13A and 13B, the percentage of seeds showing radical emergence was scored 4 d post-stratification. Germination of the seeds was monitored from 0 to 7 d on MS medium containing 150 mM NaCl and 200 mM mannitol. Two graphs (FIGS. 13A and 13B) share the same symbols: Col-0 (filled circle), pub10-1(empty triangle), 35S:PUB10 (empty circle) and 35S:mPUB10 (filled triangle). FIG. 13C: Pictures of Col-0, pub10-1 and pub10-2 seeds germinated on medium containing 150 mM NaCl were taken at 5 d after germination.

### DETAILED DESCRIPTION OF THE INVENTION

The present invention relates to the field of plant breeding and genetics, and in particular relates to recombinant DNA constructs useful in plants for conferring tolerance to drought.

Unless defined otherwise, all technical and scientific terms used herein have the same meaning as is commonly understood by one of skill in the art to which the invention belongs.

“PUB10 polypeptide” refers to an Arabidopsis thaliana polypeptide encoded by the Arabidopsis thaliana locus At1g71020. The terms “PUB10 polypeptide”, “PUB10 protein” and “PUB 10” are used interchangeably herein. The protein (SEQ ID NO:2) encoded by the gene At1g71020 is a member of the large family of plant U-box (PUB) proteins (Yee and Goring, 2009). Silencing the PUB10 gene conveys a drought tolerant phenotype. The term “PUB10” may also used herein to refer to “PUB10”, “ZM-PUB10” and “PUB10-like” unless the context dictates otherwise.

“ZM-PUB10 polypeptide” refers to a Zea mays polypeptide encoded by the Zea mays gene locus dpzm04g046470. The terms “ZM-PUB10 polypeptide”, “ZM-PUB 10 protein” and “ZM-PUB10” are used interchangeably herein. The protein (SEQ ID NO:51) encoded by the gene locus dpzm04g046470 has sequence homology with PUB10. Silencing the ZM-PUB10 gene conveys a drought tolerant phenotype.

“PUB10-like polypeptide” refers to a polypeptide having sequence homology to PUB10 and silencing the PUB-like gene conveys a drought tolerant phenotype. The terms “PUB10-like polypeptide”, “PUB10-like protein” and “PUB10-like” are used interchangeably herein.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

A “trait” refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, early seedling vigor and seedling emergence under low temperature stress.

Yield can be measured in many ways, including, for example, test weight, seed weight, seed number per plant, seed number per unit area (i.e. seeds, or weight of seeds, per acre), bushels per acre, tonnes per hectare, tonnes per acre, tons per acre and kilograms per hectare.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

The growth and emergence of maize silks has a considerable importance in the determination of yield under drought (Fuad-Hassan et al. 2008 Plant Cell Environ. 31:1349-1360). When soil water deficit occurs before flowering, silk emergence out of the husks is delayed while anthesis is largely unaffected, resulting in an increased anthesis-silking interval (ASI) (Edmeades et al. 2000 Physiology and Modeling Kernel set in Maize (eds M. E. Westgate & K. Boote; CSSA (Crop Science Society of America) Special Publication No.29. Madison, Wis. CSSA, 43-73). Selection for reduced ASI has been used successfully to increase drought tolerance of maize (Edmeades et al. 1993 Crop Science 33: 1029-1035; Bolanos & Edmeades 1996 Field Crops Research 48:65-80; Bruce et al. 2002 J. Exp. Botany 53:13-25).

Terms used herein to describe thermal time include “growing degree days” (GDD), “growing degree units” (GDU) and “heat units” (HU).

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

The term “down-regulate” and its forms, e.g. down-regulation, refers to a reduction which may be partial or complete. For example, down-regulation of a PUB10 polynucleotide in a plant or cell encompasses a reduction in expression to a level that is 99%, 95%, 90%, 85%, 80%, 75%, 70%, 65%, 60%, 55%, 50%, 45%, 40%, 35%, 30%, 25%, 20%, 15%, 10%, 5% or 0% of the expression level of the corresponding PUB10 polynucleotide in a control plant or cell. The term “reducing expression” and its forms, e.g., reduce expression, is used interchangeably with down-regulation.

A “control” or “control plant” or “control plant cell” provides a reference point for measuring changes in phenotype of a subject plant or plant cell in which genetic alteration, such as transformation, has been effected as to a polynucleotide of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the polynucleotide of interest or (e) the subject plant or plant cell itself, under conditions in which the polynucleotide of interest is not expressed.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Turning Now to the Embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present disclosure includes the following isolated polynucleotides and polypeptides:

An isolated polynucleotide comprising all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present disclosure. The polypeptide is preferably a PUB10 polypeptide. Reducing expression of a PUB10 polypeptide in a plant preferably conveys drought tolerance to the plant.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and combinations thereof. The polypeptide is preferably a PUB10 polypeptide. Reducing expression of a PUB10 polypeptide in a plant preferably conveys drought tolerance to the plant.

An isolated polynucleotide comprising all or part of (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1 or 50, and combinations thereof; or (ii) a full complement of the nucleic acid sequence of (i). Any of the foregoing isolated polynucleotides may be utilized in any recombinant DNA constructs (including suppression DNA constructs) of the present disclosure. The isolated polynucleotide preferably encodes a PUB10 polypeptide. Reducing expression of a PUB10 polypeptide in a plant preferably conveys drought tolerance to the plant.

An isolated polynucleotide comprising a nucleotide sequence, wherein the nucleotide sequence is hybridizable under stringent conditions with a DNA molecule comprising the full complement of SEQ ID NO:1 or 50. The isolated polynucleotide preferably encodes a PUB10 polypeptide. Reducing expression of a PUB10 polypeptide in a plant preferably conveys drought tolerance to the plant.

An isolated polynucleotide comprising a nucleotide sequence, wherein the nucleotide sequence is derived from SEQ ID NO:1 or 50 by alteration of one or more nucleotides by at least one method selected from the group consisting of: deletion, substitution, addition and insertion. The isolated polynucleotide preferably encodes a PUB10 polypeptide. Reducing expression of a PUB10 polypeptide in a plant preferably conveys drought tolerance to the plant.

An isolated polynucleotide comprising a nucleotide sequence, wherein the nucleotide sequence corresponds to an allele of SEQ ID NO:1 or 50.

An isolated polynucleotide that is a modified miRNA precursor in which the precursor has been modified to replace the miRNA encoding region with a sequence designed to produce a miRNA directed to SEQ ID NO:1 or 50.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NO:2 or 51. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p.6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of SEQ ID NO:1 or 50. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of SEQ ID NO:1 or 50.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and Suppression DNA Constructs:

In one aspect, the present disclosure includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and combinations thereof; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1 or 50, and combinations thereof; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide encodes all OT part of a PUB10 polypeptide. Reducing expression of a PUB10 polypeptide in a plant preferably conveys drought tolerance to the plant. The PUB10 polypeptide may be from Arabidopsis thaliana, Zea mays, Glycine max, Glycine tabacina, Glycine sofa, Glycine tomentella, Oryza sativa, Brassica napus, Sorghum bicolor, Saccharum officinarum, or Triticum aestivum.

In another aspect, the present disclosure includes suppression DNA constructs.

A suppression DNA construct may comprise at least one regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and combinations thereof, or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUB polypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1 or 50, and combinations thereof, or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest. In one embodiment, a region is derived from ZM-PUB10 and has the sequence set forth in SE! ID NO:52.

A suppression DNA construct may comprise 100, 200, 300, 400, 500, 600, 700, 800, 900 or 1000 contiguous nucleotides of the sense strand (or antisense strand) of the gene of interest, and combinations thereof. In one embodiment, the suppression DNA construct may comprises the sequence set forth in SEQ ID NO:52.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs. In one embodiment, a hairpin suppression construct comprises the sequence set forth in SEQ ID NO:52 present in both a sense and antisense orientation.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations. In one embodiment, a miRNA suppression construct comprises at least one heterologous regulatory element operably linked to a polynucleotide in which the polynucleotide is a modified plant miRNA precursor in which the precursor has been modified to replace the miRNA encoding region with a sequence designed to produce a miRNA directed to SEQ ID NO:1 or 50.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA*sequence” are used interchangeably herein and they refer to a sequence in the miRNA precursor that is highly complementary to the miRNA sequence. The miRNA and miRNA*sequences form part of the stem region of the miRNA precursor hairpin structure.

In one embodiment, there is provided a method for the suppression of a target sequence comprising introducing into a cell a nucleic acid construct encoding a miRNA substantially complementary to the target. In some embodiments the miRNA comprises about 19, 20, 21, 22, 23, 24 or 25 nucleotides. In some embodiments the miRNA comprises 21 nucleotides. In some embodiments the nucleic acid construct encodes the miRNA. In some embodiments the nucleic acid construct encodes a polynucleotide precursor which may form a double-stranded RNA, or hairpin structure comprising the miRNA.

In some embodiments, the nucleic acid construct comprises a modified endogenous plant miRNA precursor, wherein the precursor has been modified to replace the endogenous miRNA encoding region with a sequence designed to produce a miRNA directed to the target sequence. The plant miRNA precursor may be full-length of may comprise a fragment of the full-length precursor. In some embodiments, the endogenous plant miRNA precursor is from a dicot or a monocot. In some embodiments the endogenous miRNA precursor is from Arabidopsis, tomato, maize, soybean, sunflower, sorghum, canola, wheat, alfalfa, cotton, rice, barley, millet, sugar cane or switchgrass.

In some embodiments, the miRNA template, (i.e. the polynucleotide encoding the miRNA), and thereby the miRNA, may comprise some mismatches relative to the target sequence. In some embodiments the miRNA template has >1 nucleotide mismatch as compared to the target sequence, for example, the miRNA template can have 1, 2, 3, 4, 5, or more mismatches as compared to the target sequence. This degree of mismatch may also be described by determining the percent identity of the miRNA template to the complement of the target sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the target sequence.

In some embodiments, the miRNA template, (i.e. the polynucleotide encoding the miRNA) and thereby the miRNA, may comprise some mismatches relative to the miRNA-star sequence. In some embodiments the miRNA template has >1 nucleotide mismatch as compared to the miRNA-star sequence, for example, the miRNA template can have 1, 2, 3, 4, 5, or more mismatches as compared to the miRNA-star sequence. This degree of mismatch may also be described by determining the percent identity of the miRNA template to the complement of the miRNA-star sequence. For example, the miRNA template may have a percent identity including about at least 70%, 75%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% as compared to the complement of the miRNA-star sequence.

Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. AppL Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2 S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Additional promoters include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Cim1 which is specific to the nucleus of developing maize kernels. Cim1 transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one heterologous enhancer element; e.g., a 35S, nos or ocs enhancer element.

Additional promoters include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and PUB10 polypeptide genes to be used in recombinant DNA constructs (including suppression DNA constructs) and other compositions (e.g. transgenic plants, seeds and cells) and methods of the present disclosure. Examples of suitable plants for the isolation of genes and regulatory sequences and for compositions and methods of the present disclosure would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

Compositions:

A composition of the present disclosure includes a transgenic microorganism, cell, plant, and seed comprising the recombinant DNA construct (including suppression DNA construct). The cell may be eukaryotic, e.g., a yeast, insect or plant cell, or prokaryotic, e.g., a bacterial cell.

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

Particular embodiments include but are not limited to the following:

1. A plant (for example, a maize, rice or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUIB10 polypeptide, and wherein said plant exhibits increased drought tolerance when compared to a control plant not comprising said suppression DNA construct. The plant may further exhibit an alteration of at least one agronomic characteristic when compared to the control plant.

2. A plant (for example, a maize, rice or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, or (b) a full complement of the nucleic acid sequence of (a), and wherein said plant exhibits increased drought tolerance when compared to a control plant not comprising said suppression DNA construct. The plant may further exhibit an alteration of at least one agronomic characteristic when compared to the control plant.

3. A plant (for example, a maize, rice or soybean plant) comprising in its genome a suppression DNA construct comprising at least one regulatory element operably linked to all or part of (a) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1 or 50, or (b) a full complement of the nucleic acid sequence of (a), and wherein said plant exhibits increased drought tolerance when compared to a control plant not comprising said suppression DNA construct. The plant may further exhibit an alteration of at least one agronomic characteristic when compared to the control plant.

4. A plant (for example, a maize, rice or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUIB10 polypeptide, and wherein said plant exhibits an alteration of at least one agronomic characteristic when compared to a control plant not comprising said suppression DNA construct.

5. A plant (for example, a maize, rice or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, or (b) a full complement of the nucleic acid sequence of (a), and wherein said plant exhibits an alteration of at least one agronomic characteristic when compared to a control plant not comprising said suppression DNA construct.

6. A plant (for example, a maize, rice or soybean plant) comprising in its genome a suppression DNA construct comprising at least one regulatory element operably linked to all or part of (a) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:1 or 50, or (b) a full complement of the nucleic acid sequence of (a), and wherein said plant exhibits an alteration of at least one agronomic characteristic when compared to a control plant not comprising said suppression DNA construct.

7. A plant (for example, a maize, rice or soybean plant) comprising in its genome a polynucleotide (optionally an endogenous polynucleotide) operably linked to at least one heterologous regulatory element, wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and wherein said plant exhibits at least one trait selected from the group consisting of: increased drought tolerance, increased yield, increased biomass, and altered root architecture, when compared to a control plant not comprising the recombinant regulatory element. The at least one heterologous regulatory element may comprise an enhancer sequence or a multimer of identical or different enhancer sequences. The at least one heterologous regulatory element may comprise one, two, three or four copies of the CaMV 35S enhancer.

8. A plant (for example, a maize, rice or soybean plant) comprising in its genome a recombinant DNA construct comprising a polynucleotide operably linked to at least one regulatory sequence, wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, and wherein said plant exhibits increased drought tolerance when compared to a control plant not comprising said recombinant DNA construct. The plant may further exhibit an alteration of at least one agronomic characteristic when compared to the control plant.

9. A plant (for example, a maize, rice or soybean plant) comprising in its genome a recombinant DNA construct comprising a polynucleotide operably linked to at least one regulatory sequence, wherein said polynucleotide encodes all or part of a PUB10 polypeptide, and wherein said plant exhibits increased drought tolerance when compared to a control plant not comprising said recombinant DNA construct. The plant may further exhibit an alteration of at least one agronomic characteristic when compared to the control plant.

10. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1 or 50; or (b) derived from SEQ ID NO:1 or 50 by alteration of one or more nucleotides by at least one method selected from the group consisting of: deletion, substitution, addition and insertion; and wherein said plant exhibits increased tolerance to drought stress, when compared to a control plant not comprising said recombinant DNA construct. The plant may further exhibit an alteration of at least one agronomic characteristic when compared to the control plant.

11. A plant (for example, a maize, rice or soybean plant) comprising in its genome a suppression DNA construct comprising at least one regulatory element operably linked to a polynucleotide comprising a modified plant miRNA precursor in which the precursor has been modified to replace the miRNA encoding region with a sequence designed to produce a miRNA directed to SEQ ID NO:1 or 50, wherein said plant exhibits increased drought tolerance when compared to a control plant not comprising said suppression DNA construct. The plant may further exhibit an alteration of at least one agronomic characteristic when compared to the control plant.

12. Any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the PUB10 polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, or Triticum aestivum.

In any of the embodiments described herein, the recombinant DNA construct (or suppression DNA construct) may comprise at least a promoter functional in a plant as a regulatory sequence.

In any of the embodiments described herein or any other embodiments of the present disclosure, the alteration of at least one agronomic characteristic is either an increase or decrease.

In any of the embodiments described herein, the at least one agronomic characteristic may be selected from the group consisting of: abiotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, early seedling vigor and seedling emergence under low temperature stress. For example, the alteration of at least one agronomic characteristic may be an increase, e.g., in drought tolerance, yield, stay-green or biomass (or any combination thereof), or a decrease, e.g., in root lodging.

In any of the embodiments described herein, the plant may exhibit the alteration of at least one agronomic characteristic when compared, under water limiting conditions, to a control plant not comprising said recombinant DNA construct (or said suppression DNA construct).

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

“Drought” refers to a decrease in water availability to a plant that, especially when prolonged, can cause damage to the plant or prevent its successful growth (e.g., limiting plant growth or seed yield). “Water limiting conditions” refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or suppression DNA construct in its genome exhibits increased drought tolerance relative to a reference or control plant, the reference or control plant does not comprise in its genome the recombinant DNA construct or suppression DNA construct.

The terms “heat stress” and “temperature stress” are used interchangeably herein, and are defined as where ambient temperatures are hot enough for sufficient time that they cause damage to plant function or development, which might be reversible or irreversible in damage.” High temperature” can be either “high air temperature” or “high soil temperature”, “high day temperature” or “high night temperature, or a combination of more than one of these.

In one embodiment of the disclosure, the ambient temperature can be in the range of 30° C. to 36° C. In one embodiment of the disclosure, the duration for the high temperature stress could be in the range of 1-16 hours.

Typically, when a transgenic plant comprising a recombinant DNA construct or suppression DNA construct in its genome exhibits increased stress tolerance relative to a reference or control plant, the reference or control plant does not comprise in its genome the recombinant DNA construct or suppression DNA construct.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing drought tolerance in a plant, methods for evaluating drought tolerance in a plant, methods for altering an agronomic characteristic in a plant, methods for determining an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods Include But are Not Limited to the Following:

A method for transforming a cell (or microorganism) comprising transforming a cell (or microorganism) with any of the isolated polynucleotides or recombinant DNA constructs (including suppression DNA constructs) of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a recombinant DNA construct comprising a polynucleotide of the disclosure operably linked to at least one regulatory sequence, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct (including suppression DNA construct) of the present disclosure; and (b) growing the transformed host cell under conditions that are suitable for expression of the recombinant DNA construct (or suppression DNA construct) wherein expression of the recombinant DNA construct results in production of altered levels of the polypeptide of the disclosure in the transformed host cell.

A method of increasing drought tolerance in a plant, comprising: (a) introducing into a regenerable plant cell a recombinant DNA construct comprising a polynucleotide operably linked to at least one regulatory sequence (for example, a promoter functional in a plant), wherein the polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51; and (b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased drought tolerance when compared to a control plant not comprising the recombinant DNA construct.

A method of increasing drought tolerance, the method comprising: (a) introducing into a regenerable plant cell a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1 or 50; or (b) derived from SEQ ID NO:1 or 50 by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased drought tolerance, when compared to a control plant not comprising the recombinant DNA construct.

A method of increasing drought tolerance in a plant, comprising: (a) introducing into a regenerable plant cell a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, or (ii) a full complement of the nucleic acid sequence of (a)(i); and (b) regenerating a transgenic plant from the regenerable plant cell after step (a), wherein the transgenic plant comprises in its genome the suppression DNA construct and exhibits increased drought tolerance when compared to a control plant not comprising the suppression DNA construct. The method may further comprise (c) obtaining a progeny plant derived from the transgenic plant, wherein said progeny plant comprises in its genome the suppression DNA construct and exhibits increased drought tolerance when compared to a control plant not comprising the suppression DNA construct.

A method of increasing drought tolerance in a plant, comprising: (a) introducing into a regenerable plant cell a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUB10 polypeptide; and (b) regenerating a transgenic plant from the regenerable plant cell after step (a), wherein the transgenic plant comprises in its genome the suppression DNA construct and exhibits increased drought tolerance when compared to a control plant not comprising the suppression DNA construct. The method may further comprise (c) obtaining a progeny plant derived from the transgenic plant, wherein said progeny plant comprises in its genome the suppression DNA construct and exhibits increased drought tolerance when compared to a control plant not comprising the suppression DNA construct.

A method of increasing drought tolerance in a plant, comprising: (a) introducing into a regenerable plant cell a suppression DNA construct comprising at least one regulatory sequence (for example, a promoter functional in a plant) operably linked to a polynucleotide comprising a modified plant miRNA precursor in which the precursor has been modified to replace the miRNA encoding region with a sequence designed to produce a miRNA directed to SEQ ID NO:1 or 50, and (b) regenerating a transgenic plant from the regenerable plant cell after step (a), wherein the transgenic plant comprises in its genome the suppression DNA construct and exhibits increased drought tolerance when compared to a control plant not comprising the suppression DNA construct. The method may further comprise (c) obtaining a progeny plant derived from the transgenic plant, wherein said progeny plant comprises in its genome the suppression DNA construct and exhibits increased drought tolerance when compared to a control plant not comprising the suppression DNA construct.

A method of selecting for (or identifying) increased drought tolerance in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51; (b) obtaining a progeny plant derived from said transgenic plant, wherein the progeny plant comprises in its genome the recombinant DNA construct; and (c) selecting (or identifying) the progeny plant with increased drought tolerance compared to a control plant not comprising the recombinant DNA construct.

In another embodiment, a method of selecting for (or identifying) increased drought tolerance in a plant, comprising: (a) obtaining a transgenic plant, wherein the transgenic plant comprises in its genome a recombinant DNA construct comprising a polynucleotide operably linked to at least one regulatory element, wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51; (b) growing the transgenic plant of part (a) under conditions wherein the polynucleotide is expressed; and (c) selecting (or identifying) the transgenic plant of part (b) with increased drought tolerance compared to a control plant not comprising the recombinant DNA construct.

A method of selecting for (or identifying) increased drought tolerance in a plant, the method comprising: (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1 or 50; or (ii) derived from SEQ ID NO:1 or 50 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant with increased drought tolerance, when compared to a control plant not comprising the recombinant DNA construct.

A method of selecting for (or identifying) increased drought tolerance in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, or (ii) a full complement of the nucleic acid sequence of (a)(i); (b) obtaining a progeny plant derived from said transgenic plant, wherein the progeny plant comprises in its genome the suppression DNA construct; and (c) selecting (or identifying) the progeny plant with increased drought tolerance compared to a control plant not comprising the suppression DNA construct.

A method of selecting for (or identifying) increased drought tolerance in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUB polypeptide; (b) obtaining a progeny plant derived from the transgenic plant, wherein the progeny plant comprises in its genome the suppression DNA construct; and (c) selecting (or identifying) the progeny plant for drought tolerance compared to a control plant not comprising the suppression DNA construct.

A method of selecting for (or identifying) increased drought tolerance in a plant, comprising (a) obtaining a transgenic plant, wherein the transgenic plant comprises in its genome a suppression DNA construct comprising at least one regulatory sequence (for example, a promoter functional in a plant) operably linked polynucleotide comprising a modified plant miRNA precursor in which the precursor has been modified to replace the miRNA encoding region with a sequence designed to produce a miRNA directed to SEQ ID NO:1 or 50; (b) obtaining a progeny plant derived from said transgenic plant, wherein the progeny plant comprises in its genome the suppression DNA construct; and (c) selecting (or identifying) the progeny plant with increased drought tolerance compared to a control plant not comprising the suppression DNA construct.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared, optionally under water limiting conditions, to a control plant not comprising the recombinant DNA construct. The polynucleotide preferably encodes a PUB10 polypeptide. Reducing expression of a PUB10 polypeptide in a plant preferably conveys drought tolerance to the plant.

In another embodiment, a method of selecting for (or identifying) an alteration of at least one agronomic characteristic in a plant, comprising: (a) obtaining a transgenic plant, wherein the transgenic plant comprises in its genome a recombinant DNA construct comprising a polynucleotide operably linked to at least one regulatory element, wherein said polynucleotide encodes all or part of a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, wherein the transgenic plant comprises in its genome the recombinant DNA construct; (b) growing the transgenic plant of part (a) under conditions wherein the polynucleotide is expressed; and (c) selecting (or identifying) the transgenic plant of part (b) that exhibits an alteration of at least one agronomic characteristic when compared to a control plant not comprising the recombinant DNA construct. Optionally, said selecting (or identifying) step (c) comprises determining whether the transgenic plant exhibits an alteration of at least one agronomic characteristic when compared, under water limiting conditions, to a control plant not comprising the recombinant DNA construct. The at least one agronomic trait may be yield, biomass, or both and the alteration may be an increase.

A method of selecting for (or identifying) an alteration of an agronomic characteristic in a plant, comprising (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1 or 50; or (ii) derived from SEQ ID NO:1 or 50 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared, optionally under water limiting conditions, to a control plant not comprising the recombinant DNA construct. The polynucleotide preferably encodes a PUB10 polypeptide. Reducing expression of a PUB10 polypeptide in a plant preferably conveys drought tolerance to the plant.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO:2 or 51, or (ii) a full complement of the nucleic acid sequence of (i); (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under water limiting conditions, to a control plant not comprising the suppression DNA construct.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to said all or part of a sense strand or antisense strand from which said region is derived, and wherein said target gene of interest encodes a PUB10 polypeptide; (b) obtaining a progeny plant derived from said transgenic plant, wherein the progeny plant comprises in its genome the suppression DNA construct; and (c) selecting (or identifying) the progeny plant that exhibits an alteration in at least one agronomic characteristic when compared, optionally under water limiting conditions, to a control plant not comprising the suppression DNA construct. Reducing expression of a PUB10 polypeptide in a plant preferably conveys drought tolerance to the plant.

A method of selecting for (or identifying) an alteration of an agronomic characteristic in a plant, comprising (a) obtaining a transgenic plant, wherein the transgenic plant comprises in its genome a suppression DNA construct comprising at least one regulatory sequence (for example, a promoter functional in a plant) operably linked polynucleotide comprising a modified plant miRNA precursor in which the precursor has been modified to replace the miRNA encoding region with a sequence designed to produce a miRNA directed to SEQ ID NO:1 or 50; (b) obtaining a progeny plant derived from said transgenic plant, wherein the progeny plant comprises in its genome the suppression DNA construct; and (c) selecting (or identifying) the progeny plant with increased drought tolerance compared to a control plant not comprising the suppression DNA construct.

A method of producing a plant that exhibits at least one trait selected from the group consisting of: increased drought tolerance, increased yield, increased biomass, and altered root architecture, wherein the method comprises growing a plant from a seed comprising a recombinant DNA construct (or suppression DNA construct), wherein the recombinant DNA construct (or suppression DNA construct) comprises a polynucleotide operably linked to at least one heterologous regulatory element, wherein the polynucleotide encodes all or part of a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:2 or 51, wherein the plant exhibits at least one trait selected from the group consisting of: increased drought tolerance, increased yield, increased biomass, and altered root architecture, when compared to a control plant not comprising the recombinant DNA construct (or suppression DNA construct). The polynucleotide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing a seed, the method comprising: (a) crossing a first plant with a second plant, wherein at least one of the first plant and the second plant comprises a recombinant DNA construct (or suppression DNA construct), wherein the recombinant DNA construct (or suppression DNA construct) comprises a polynucleotide operably linked to at least one heterologous regulatory element, wherein the polynucleotide encodes all or part of a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:2 or 51; and (b) selecting a seed of the crossing of step (a), wherein the seed comprises the recombinant DNA construct (or suppression DNA construct). A plant grown from the seed may exhibit at least one trait selected from the group consisting of: increased drought tolerance, increased yield, increased biomass, and altered root architecture, when compared to a control plant not comprising the recombinant DNA construct (or suppression DNA construct). The polynucleotide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing seed (for example, seed that can be sold as a drought tolerant product offering) comprising any of the preceding methods, and further comprising obtaining seeds from said progeny plant, wherein said seeds comprise in their genome said recombinant DNA construct (or suppression DNA construct).

A method of producing oil or a seed by-product, or both, from a seed, the method comprising extracting oil or a seed by-product, or both, from a seed that comprises a recombinant DNA construct (or suppression DNA construct), wherein the recombinant DNA construct (or suppression DNA construct) comprises a polynucleotide operably linked to at least one heterologous regulatory element, wherein the polynucleotide encodes all or part of a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:2 or 51. The seed may be obtained from a plant that comprises the recombinant DNA construct (or suppression DNA construct), wherein the plant exhibits at least one trait selected from the group consisting of: increased drought tolerance, increased yield, increased biomass, and altered root architecture, when compared to a control plant not comprising the recombinant DNA construct (or suppression DNA construct). The polynucleotide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass. The oil or the seed by-product, or both, may comprise the recombinant DNA construct (or suppression DNA construct).

Methods of isolating seed oils are well known in the art: (Young et al., Processing of Fats and Oils, In The Lipid Handbook, Gunstone et al., eds., Chapter 5 pp 253 257; Chapman & Hall: London (1994)). Seed by-products include but are not limited to the following: meal, lecithin, gums, free fatty acids, pigments, soap, stearine, tocopherols, sterols and volatiles.

One may evaluate altered root architecture in a controlled environment (e.g., greenhouse) or in field testing. The evaluation may be under limiting or non-limiting water conditions. The evaluation may be under simulated or naturally-occurring low or high nitrogen conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not comprising the recombinant DNA construct (or suppression DNA construct).

The use of a recombinant DNA construct (or suppression DNA construct) for producing a plant that exhibits at least one trait selected from the group consisting of: increased drought tolerance, increased yield, increased biomass, and altered root architecture, when compared to a control plant not comprising said recombinant DNA construct (or suppression DNA construct), wherein the recombinant DNA construct (or suppression DNA construct) comprises a polynucleotide operably linked to at least one heterologous regulatory element, wherein the polynucleotide encodes all or part of a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:2 or 51. The polynucleotide may be expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

In any of the preceding methods or any other embodiments of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the at least one agronomic characteristic may be selected from the group consisting of: abiotic stress tolerance, greenness, stay-green, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, amino acid content in a vegetative tissue, total plant protein content, fruit protein content, seed protein content, protein content in a vegetative tissue, drought tolerance, nitrogen stress tolerance, nitrogen uptake, root lodging, root mass, harvest index, stalk lodging, plant height, ear height, ear length, salt tolerance, early seedling vigor and seedling emergence under low temperature stress. The alteration of at least one agronomic characteristic may be an increase, e.g., in drought tolerance, yield, stay-green or biomass (or any combination thereof), or a decrease, e.g., in root lodging.

In any of the preceding methods or any other embodiments of methods of the present disclosure, the plant may exhibit the alteration of at least one agronomic characteristic when compared, under water limiting conditions, to a control plant not comprising said recombinant DNA construct (or said suppression DNA construct).

In any of the preceding methods or any other embodiments of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

Additional Embodiments of the Present Invention Include:
use of PUB11 in any of the embodiments described herein, in which SEQ ID NO:3 is included with SEQ ID NOs:1 and 50 or in which SEQ ID NO:4 is included with SEQ ID NOs:2 and 51 or in which PUB11 is used in context as is PUB10;
use of the PUB10 promoter for making nucleic acid constructs and the nucleic acid constructs in accordance with techniques well known in the art of plant molecular biology;
the C249A allele of AT-PUB10, and the corresponding mutation in ZM-PUB10 and nucleic acid constructs containing the same;
use of CRISPR to generate a C249A mutation in Arabidopsis and/or the corresponding mutation in the maize gene encoding ZM-PUB10; and
drought tolerant plants containing the C249A allele of AT-PUB10 or the corresponding mutant allele in maize, preferably plants that are homozygous for these alleles.

For example, in one embodiment, a reduction in expression of the endogenous PUB10 gene, PUB11 gene, or both may be caused by sense suppression, antisense suppression, miRNA suppression, ribozymes, or RNA interference, or the reduction may be caused by a mutation in the endogenous PUB10 gene, PUB11 gene, or both. The mutation may arise from insertional mutagenesis, such as but not limited to transposon mutagenesis, or may be caused by zinc finger nuclease, Transcription Activator-Like Effector Nuclease (TALEN), CRISPR or meganuclease technology.

The proteins of the CRISPR (clustered regularly interspaced short palindromic repeat) system are examples of DNA-binding and DNA-nuclease domains. The expression levels of an endogenous gene, or the activity of the corresponding endogenous polypeptide can be reduced by introducing mutations through CRISPRfCas9 system. The bacterial CRISPR/Cas system involves the targeting of DNA with a short, complementary single stranded RNA (CRISPR RNA or crRNA) that localizes the Cas9 nuclease to the target DNA sequence (Burgess D J (2013) Nat Rev Genet 14:80; PCT Publication No. WO20141127287). The crRNA can bind on either strand of DNA and the Cas9 will cleave the DNA making a double-strand break (DSB).

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et aL, 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Green and Sambrook, 2012, Molecular Cloning, 4th Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, N.Y., 1992); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Fire et al., RNA Interference Technology: From Basic Science to Drug Development, Cambridge University Press, Cambridge, 2005; Schepers, RNA Interference in Practice, Wiley-V C H, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Materials and Methods

Plant materials: Arabidopsis thaliana ecotype Columbia (Col-0) and pub10-1, pub10-2, pub11-1 mutant plants were used in this study. The pub10-1, (SALK_017111), pub10-2 (SALK_063407) and pub11 (SALK_029828) T-DNA insertion lines were obtained from the ABRC. Double pub10 pub11 mutants were generated by crossing pub10-1 with pub11-1.

Transgenic plants overexpressing myc-PUB10 and myc-PUB10 (C249A) were generated by cloning cDNA into pBA-6myc-DC vector under the control of a CaMV 35S promoter. XVE:myc-PUB10 and XVE:myc-PUB10 (C249A) were constructed by cloning into pER8 vector. Transgenic plants were obtained by the floral dip transformation method (Zhang XR et al., 2006). In addition, we use β-estradiol (Sigma) to induce myc-PUB10 or myc-PUB10 (C249A) expression in transgenic Arabidopsis thaliana seedlings carrying XVE:myc-PUB10 and XVE:myc-PUB10 (C249A).

ABA, β-estradiol, MG132 and cycloheximide treatments: To assay ABA effects on seed germination, seeds were plated onto MS plates with ABA (Sigma). The plates were transferred to 16 h light/8 h dark at 22° C. under white fluorescent light (70 μmol·m⁻²·s^{˜1}) Germination of the seeds was monitored from 0 to 7 d.

For root growth inhibition assays, seeds were germinated on MS plates in a growth room under 16 h light/8 h dark at 22° C. with a light intensity of 70 μmol·m⁻²·s⁻¹. After 4 d, seedlings were transferred onto square MS plates with 5 μM ABA (sigma), and the pates were placed vertically. Root length was measured 2 to 3 weeks after germination.

Transgenic Arabidopsis seedlings expressing either XVE:myc-PUB10, XVE:myc-PUB10 (C249A), 35S:myc-MYC2/XVE:HA-PUB10 or 35S:myc-MYC2/XVE:HA-PUB10 (C249A) were germinated and grown on selective media for 2 or 3 weeks (16 h light/8 h dark photoperiod) before being transfer to liquid MS medium and treated with MG132 or MG132 (EMD millipore) plus β-estradiol for 12 h. For post-transfer analysis, seedlings treated as above were washed three times with MS liquid medium, and then transferred to fresh MS liquid medium containing 1 mM cycloheximide (CHX) to block protein synthesis. Treated seedlings were collected for Western blot and real time RT-PCR analyses.

GUS staining: For promoter-GUS staining, the promoter sequence of PUB10 (˜2.2 kb; SEQ ID NO:7), PUB11 (3 kb; SEQ ID NO:8) and MYC2 (3 kb; SEQ ID NO:9) was cloned into pKGWFS7 vector to obtain the PUB10-GUS-GFP, PUB11-GUS-GFP and MYC2-GUS-GFP fusion, respectively. Gus staining was performed as previously described in Senecoff et al.

Yeast two-hybrid assays: The Matchmarker GAL4-bases two-hybrid system (Clontech) was used to perform yeast two-hybrid assays. The cDNAs encoding full length MYC2 and UBCs were cloned into pGAD424 vector (Clontech) to generate activation domain (AD) constructs. Full-length PUB10 and PUB11 cDNA or their deletion derivatives were ligated into pGBT9 vector (Clontech) to generate binding domain (BD) constructs. All constructs and empty vector controls were transformed into yeast stain AH109 by the modified lithium acetate method. Yeast transformants were screened on the selective medium SD/-Leu/-Trp/-His with 20 mM 3-amino-1,2,4,-triazole (3-AT) to test for protein interactions.

Preparation of recombinant proteins: Full length cDNA encoding MYC2 were amplified by PCR and cloned into pGEX-DC-HA to generate the coding sequence for GST-MYC2-HA. cDNAs for full-length PUB10 and its deletion derivatives were amplified by PCR and inserted into pMAL-DC-6myc to generate MBP-PUB10-6myc (full length PUB10 is SEQ ID NO:2), MBP-PUB10 (C249A)-6myc (dominant negative mutant), MBP-PUB10 (UND)-6myc (PUB10-(UND) is 1-240 aa of SEQ ID NO:2), MBP-PUB10 (U-box)-6myc (PUB10 (U-box) is 240-320 aa of SEQ ID NO:2), MBP-PUB10 (ARM)-6myc (PUB10 (ARM) is 320-628 aa of SEQ ID NO:2). cDNAs encoding full-length UBCs were amplified by PCR and cloned into pET-28a (+) (Novagen) to generate the sequences for various 6His-UBCs. cDNA sequences for UBCs 1, 2, 3, 5, 8, 10, 11, 16, 18, 22, 24, 27, 28, 29, 31, 33, 34 and 36 are set forth in SEQ ID NOs:10-27, respectively. The UBC protein sequences encoded by the UBC cDNA sequences can be readily determined using conventional translation programs. The protein sequences (UBCs, PUB10, PUB11 and MYC2) can further be encoded by DNA sequences that take into consideration the genetic code.

All vectors expressing recombinant proteins were transformed into E. coli BL21 cells and fusion protein expression was induced by isopropyl-β-D-thiogalactoside (IPTG). For GST fusion protein purification, treated cells were lysed in PBS buffer, pH7.4 containing 1 mM dithiothreitol (DTT) and a protease inhibitor cocktail (Roche). GST-tagged proteins were purified on glutathione sepharose™ R10-Flammable (GE Health) and eluted with a buffer containing 50 mM Tris-HCl pH 8.0 and 10 mM glutathione. For MBP fusion protein purification, treated cells were lysed in Column buffer containing 20 mM Tris-HCl , pH7.4, 200 mM NaCl, 1 mM dithiothreitol (DTT) and a protease inhibitor cocktail (Roche). MBP-tagged proteins were purified on amylose resin (New England Biolabs) and eluted with a Column buffer containing 10 mM maltose. For His-tagged fusion protein purification, treated cells were lysed in 50 mM sodium phosphate buffer, pH 8.0, 300 mM NaCl, 1% Triton X-100 and 2 mM PMSF. Tagged proteins were purified on Ni²⁺-nitrilotriacetate (Ni²⁺-NTA) resins (Qiagen) and eluted using buffer containing 50 mM sodium phosphate buffer, pH 8.0, 300 mM NaCl and 250 mM imidazole.

In vitro binding and ubiquitination assays: For in vitro binding assays, 2 μg of bait MBP-fusion protein (full length PUB10, or dominant negative mutant PUB C249A, or its deletion derivatives) and 2 μg of prey protein (GST-MYC2) were added into 1 ml binding buffer (50 mM Tris-HCl pH 7.5, 100 mM NaCl, 0.5% Triton X-100, 0.5 mM β-mercaptoethanol and 2% proteinase inhibiter cocktail) and incubated with amylose resin beads at 25° C. for 2 h. After incubation, beads were washed 6 times with fresh binding buffer. Pull-down proteins were separated on 8% SDS-polyacrylamide gels and detected by Western blotting using anti-GST antibody (Santa Cruz Biotechnology).

For in vitro ubiquitination, each assay (30 μl ) contained 100 ng rabbit E1 (Boston Biochem), 200 ng E2 (Human UbcH5b or Arabidopsis AtUBCs), 3 μg His₆-ubiquitin (Sigma), 1 μg fresh purified E3 (MBP-PUB10 or PUB11-6myc) and 1 μg protein substrate (GST-MYC2-3HA). Reactions were incubated at 30° C. for 3 h. The reaction mixtures were analyzed on 8% SDS-polyacrylamide gels. Ubiquitinated MBP-PUB10, MBP-PUB11 or GST-MYC2 proteins were analyzed by Western blots using anti-MBP or anti-GST.

Bimolecular Fluorescence Complementation (BiFC) Assays: Full-length cDNA encoding MYC2 and PUB10 (C249A) were cloned into the BiFC vectors to test for proteins interaction in vivo. Recombinant plasmids encoding PUB10 (C249A)-cYFP and MYC2-nYFP fusions were transformed into competent Agrobacterium (strain ABI) cells which were then cultured. Agrobacterial cells were collected and suspended in a solution containing 10 mM MgCl₂ and 150 uM acetosyringone in the presence of MG132 (50 μM), and then kept at 25° C. for at least 3 h without shaking. Agrobacterial suspension was infiltrated into leaves of Nicotiana benthaminana and after 2 days infiltrated plants were analyzed by microscopy.

Protein extraction and western blotting: Arabidopsis seedlings were harvested and ground in an extraction buffer (50 mM Tris-HCl pH8.0, 100 mM NaCl, 10 mM MgCl2, 0.1% IGEPAL CA6300, 0.5 mM PMSF and a protease inhibitor cocktail) and the extract was clarified by centrifugation. Equivalent amount of protein extracts was separated on 8% SDS-polyacrylamide gels and then transferred to a polyvinylidene difluoride (PVDF) membranes (Millipore) at 4° C. Western analysis was performed with anti-HA, anti-Myc (Santa Cruz Biotechnology) or anti-Tubulin (Sigma) primary antibodies and Horseradish Peroxidase (HRP)-linked rabbit or mouse secondary antibodies (GE Health UK limited). An ECL kit (GE Health UK limited) was used for western blot signal detection. Tubulin levels were used as a loading control.

RNA extraction and Real-time PCR analysis: Total RNA was extracted from Arabidopsis seedlings using the RNeasy plant mini kit (Qiagen). Reverse transcription was performed using 2 μg of each total RNA and oligo (dT) primers by the SuperScript III RT kit (Invitrogen). The cDNA was mixed with SYBR premix Ex Taq (TaKaRa) and gene-specific primers in a Bio-Rad CFX96 real-time PCR system. Primer sequences for real-time PCR are presented in Table 1.

TABLE 1
Oligonucleotide Primer Sequences

| | | De- |
|---|---|---|
| | | scrip- |
| Name | Sequence (5′→3′) (SEQ ID NO:) | tion |
| PUB10_ | GACAAGGGTACCATGGCTGGTGGAGCTATCACTC | Clon- |
| CDS-F | CC (28) | ing |
| PUB10_ | GACAAGGCGGCCGCGAGTGAACCTAATTTTCGGG | |
| CDS-R | (29) | |
| PUB10_ | AGAGGACTTTCTTGCTCCAATCTCTCTGG | Muta- |
| C249A- | (30) | gene- |
| F | | sis |
| PUB10_ | CCAGAGAGATTGGAGCAAGAAAGTCCTCT | |
| C249A- | (31) | |
| R | | |
| PUB10_ | GACAAGGGTACCGAGATTGTTTGCTAAGAAAATGG | Clon- |
| promo- | (32) | ing |
| ter-F | | |
| PUB10_ | GACAAGGCGGCCGCTACGCCGTCTCACACGGCGG | |
| promo- | (33) | |
| ter-R | | |
| PUB11_ | GACAAGGGTACCATGGCCGGAGGAATCGTCTCACC | Clon- |
| CDS-F | (34) | ing |
| PUB11_ | GACAAGGCGGCCGCTTGGCATGCTTTACGAAGAA | |
| CDS-R | (35) | |
| PUB11_ | GGTTGATTTTCTTGCTCCGGTGTCGCTTG | Muta- |
| C247A- | (36) | gene- |
| F | | sis |
| PUB10_ | CAAGCGACACCGGAGCAAGAAAATCAACC | |
| C247A- | (37) | |
| R | | |
| PUB11_ | GACAAGGGTACCCTCTACCCTCCAGTTTCTAGCT | Clon- |
| promo- | CC (38) | ing |
| ter-F | | |
| PUB11_ | GACAAGGCGGCCGCTACGCCGTCGCCGATCAACC | |
| promo- | (39) | |
| ter-R | | |
| MYC2_ | GACAAGGGTACCATGACTGATTACCGGCTACAACC | Clon- |
| CDS-F | (40) | ing |
| MYC2_ | GACAAGGCGGCCGCACCGATTTTTGAAATCAAAC | |
| CDS-R | (41) | |
| MYC2_ | GACAAGGGTACCCTAGTGGCGTCACCCCCAAAG | Clon- |
| promo- | (42) | ing |
| ter-F | | |
| MYC2_ | GACAAGCTCGAGTCCATAAACCGGTGACCGGT | |
| promo- | (43) | |
| ter-R | | |
| 6xmyc- | ACCTCACCATGGAGCAAAAGC | Real |
| F | (44) | time |
| MYC2-R | AGATTCATCGTTGGTTGTAGCCG | RT- |
| | (45) | PCR |
| SALK_ | AGAAGGATTGTTCCGATCTCG | Geno- |
| 017111- | (46) | typing |
| LP | | |
| SALK_ | ACACATCAAAGTTTAGAGAGCTCC | |
| 017111- | (47) | |
| RP | | |
| SALK_ | TGGTGGAGCTATCACTCCCG | Geno- |
| 063407- | (48) | typing |
| LP | | |
| SALK_ | TGGTGGAGCTATCACTCCCG | |
| 063407- | (49) | |
| RP | | |

### Example 2

### PUB10 Interacts With MYC2 in Yeast Cell and in vitro

In order to investigate the biological role of PUB10 in Arabidopsis, a yeast two hybrid assays was performed using PUB10 as bait to interrogate a small library of prey comprising of about 1,500 transcription factors encoded by the Arabidopsis genome (Mitsuda et al., 2010). Preliminary experiments uncovered several candidate proteins that interacted with PUB10 in yeast cells. The strongest among these was MYC2, a basic helix-loop-helix (bHLH) protein, which was further investigated.

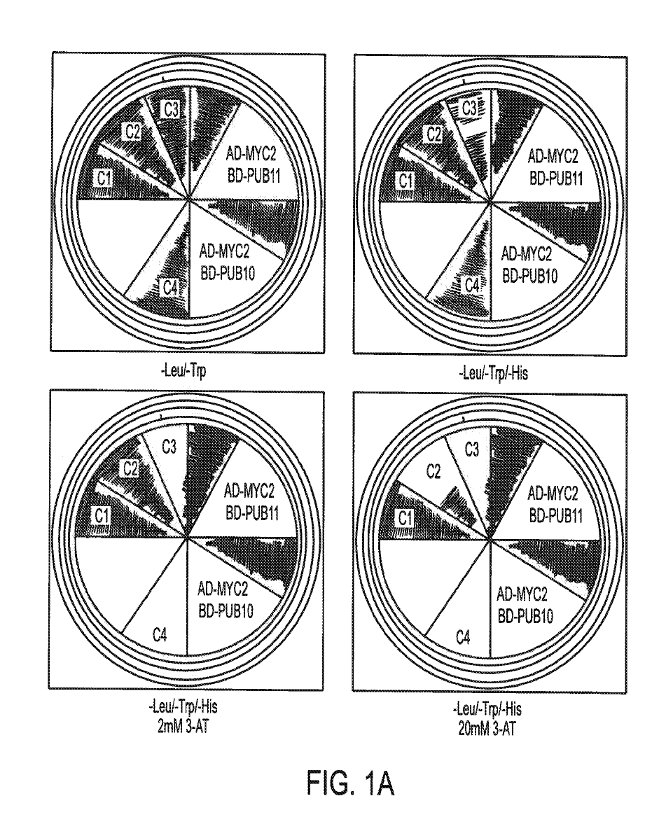

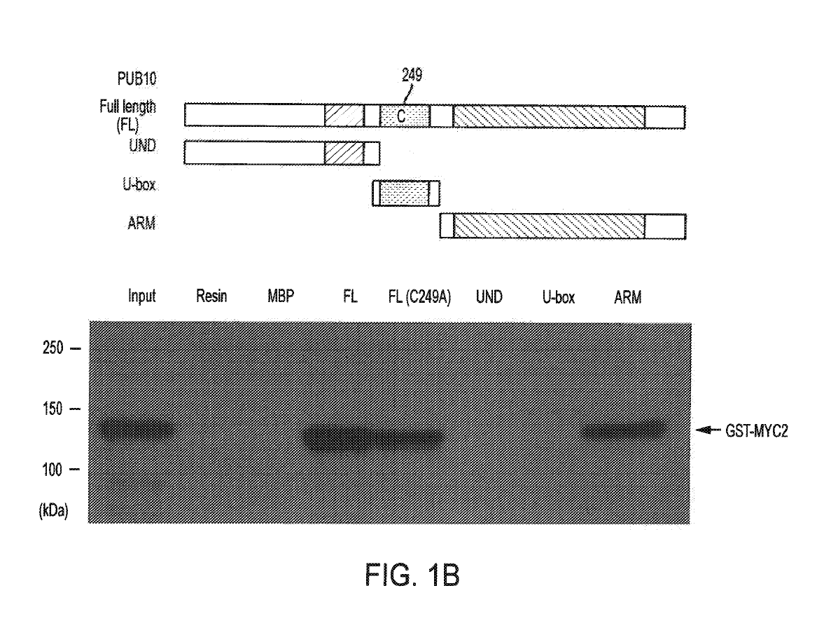

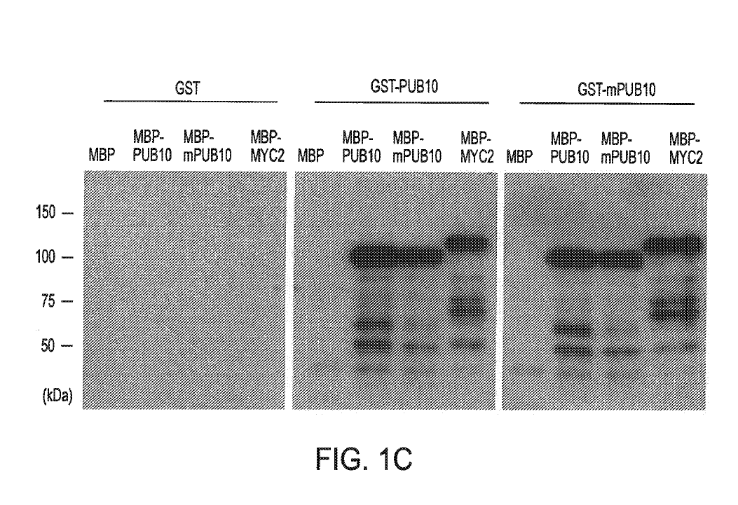

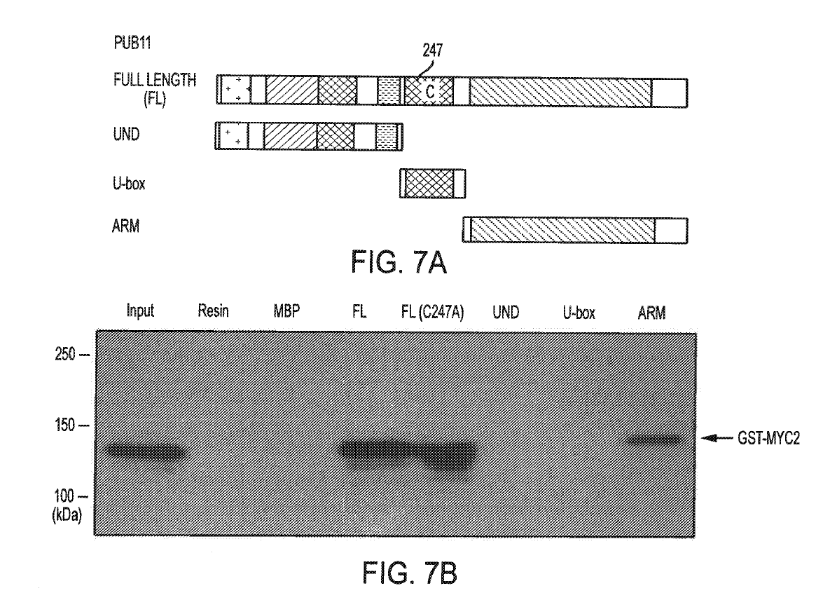

FIG. 1A shows that MYC2 interacted with PUB10 and PUB11 in yeast two hybrid assays performed under stringent conditions. To confirm this result, full-length WT PUB10 protein purified from E. coli extracts was tested for its capacity to bind to MYC2. Indeed, full-length WT PUB10 was able to bind to MYC2 in vitro (FIG. 1B). Following the first demonstration of a PUB protein to have E3 ligase activity several PUB proteins have been shown to have the same activity. Although PUB proteins do not possess a RING motif, certain cysteine residues are essential for E3 ligase activity. A PUB10 mutant with a C249A (cysteine to alanine) mutation and several PUB10 deletion derivatives (FIG. 1B) were generated and tested for their capacity to bind to MYC2 in vitro. FIGS. 1B and 1C show that MYC2 binding was not compromised by the C249 mutation. It was found that cysteine mutant of PUB10 (mPUB10) forms a dimer with WT PUB10 and interacts with MYC2 (FIG. 1C). Analysis of PUB10 deletion derivatives localized the MYC2 binding region to the C-terminal fragment of PUB10, which contains the ARM repeats. Similar results were obtained with PUB11, the closet homolog of PUB10 (FIGS. 7A and 7B).

### Example 3

### PUB10 Interacts with Specific UBCs and Polyubiquitinates MYC2 in vitro

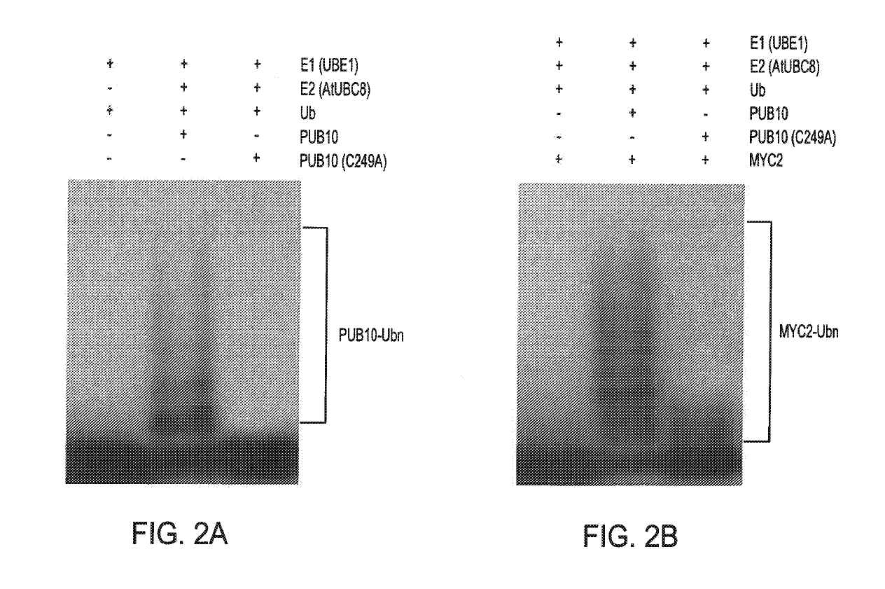

The activity of PUB10 was examined. PUB10 protein purified from E. coli extracts was used as a source of E3 enzymes for in vitro ubiquitination reactions. FIG. 2A shows that PUB10 was able to perform autoubiquitination using the Arabidopsis AtUBC8 as E2 and activity was compromised by the C249A mutation.

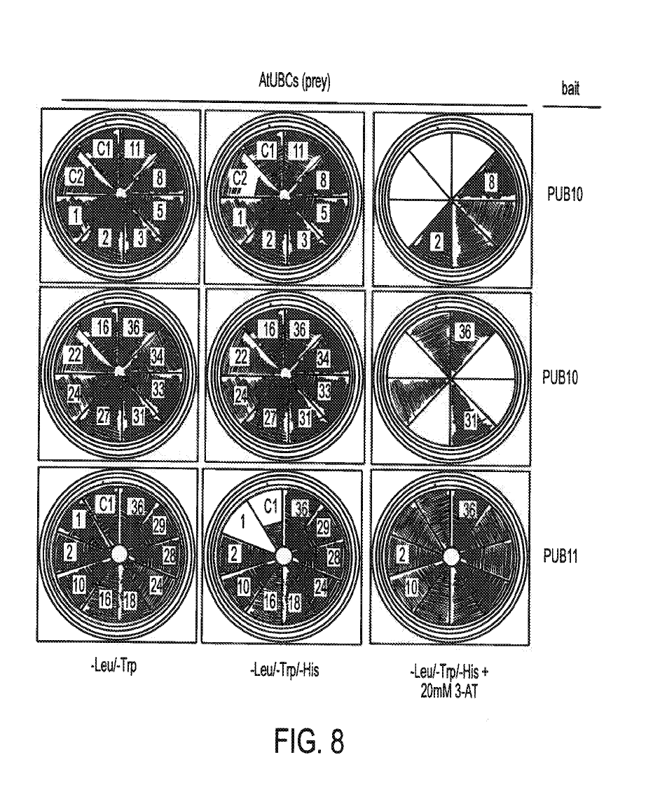

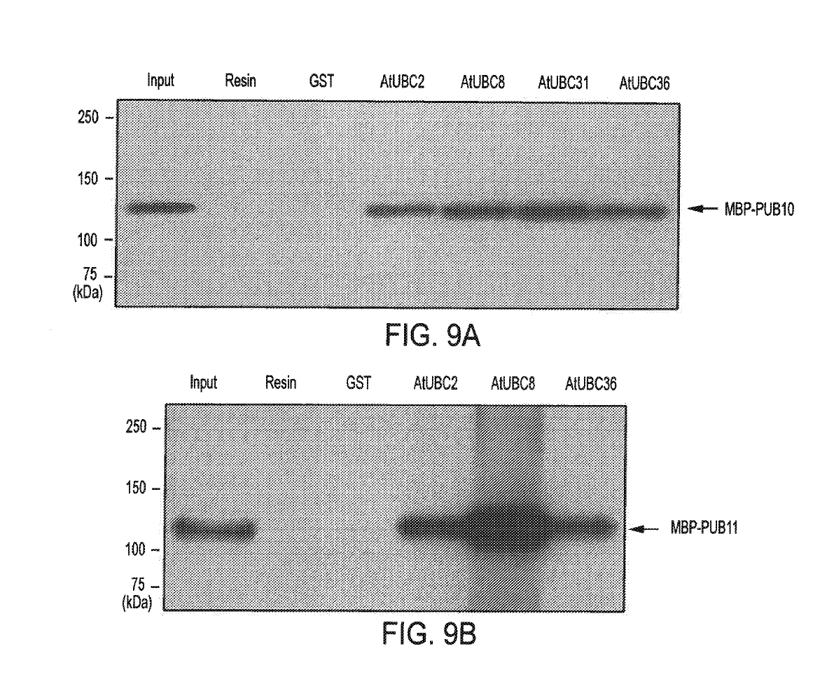

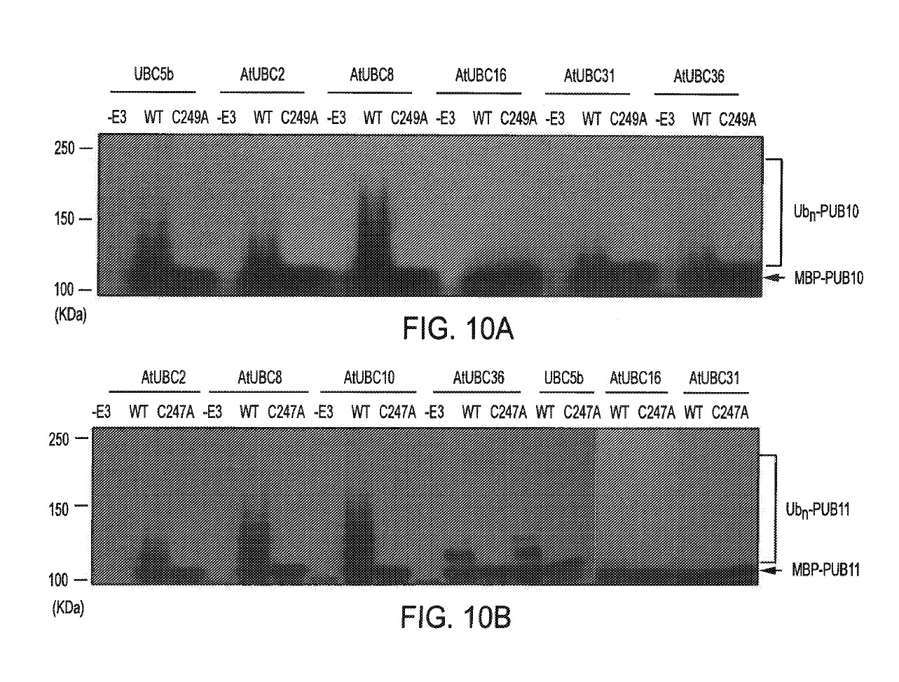

To identify other interacting E2s, yeast two hybrid assays were performed using PUB10 and PUB11 as baits and 35 Arabidopsis UBCs as preys (Kraft et al., 2005). It was found that in addition to UBC8, PUB10 and PUB11 interacted with at least 3 other Arabidopsis UBCs (#2, 31 & 36) in yeast cells and in vitro (FIG. 8 and FIG. 9A and 9B). Each of these 4 UBCs was capable of supporting auto-ubiquitination of PUB10 and PUB11 in vitro although with varying degrees of activity (FIGS. 10A and 10B). The highest activity was obtained when UBC8 served as the ubiquitin conjugating enzyme.

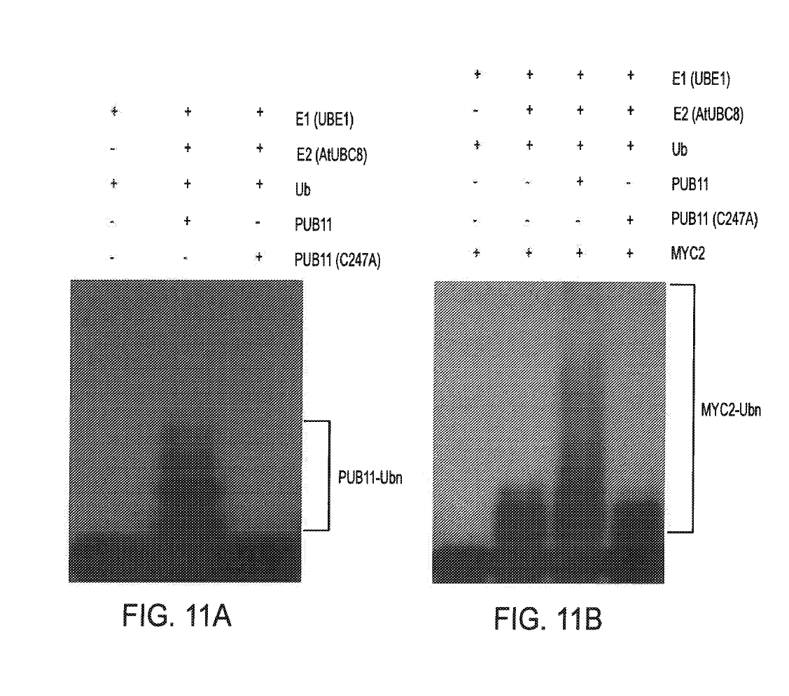

The association of PUB10 with MYC2 suggested that the latter may be a substrate of the PUB10 E3 ligase. To examine this possibility, UBC8 and PUB10 were used as the E2 and E3 enzyme, respectively. FIGS. 2A and 2B show that PUB10 has a ubiquitin E3 ligase activity and MYC2 was indeed poly-ubiquitinated by PUB10 in vitro. The ubiquitination activity was greatly reduced when cysteine 249 was mutated to alanine 249, indicating the importance of this amino acid in maintaining the E3 activity. Similar results were obtained with wild type PUB11 and PUB11 (C247A) mutant (FIGS. 11A and 11B).

### Example 4

### ABA Triggers PUB10 Association with Nuclear MYC2

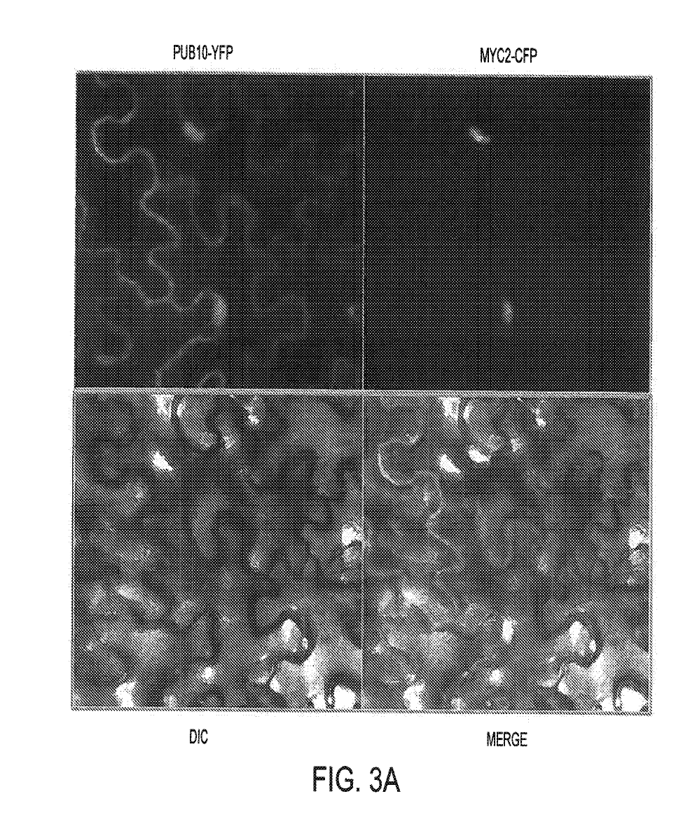

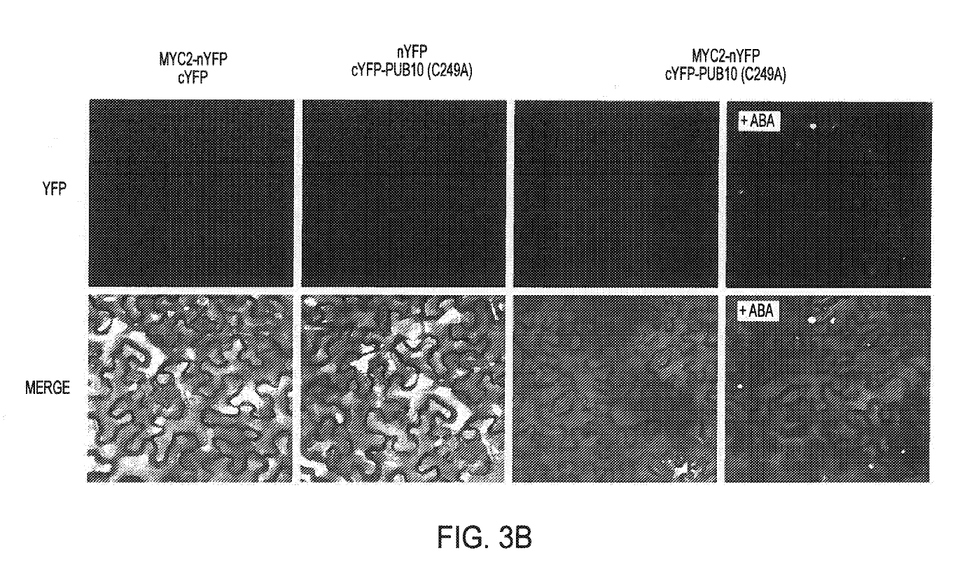

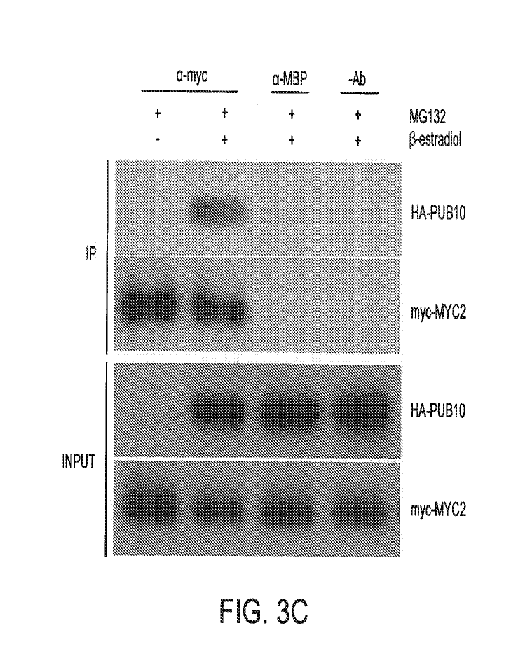

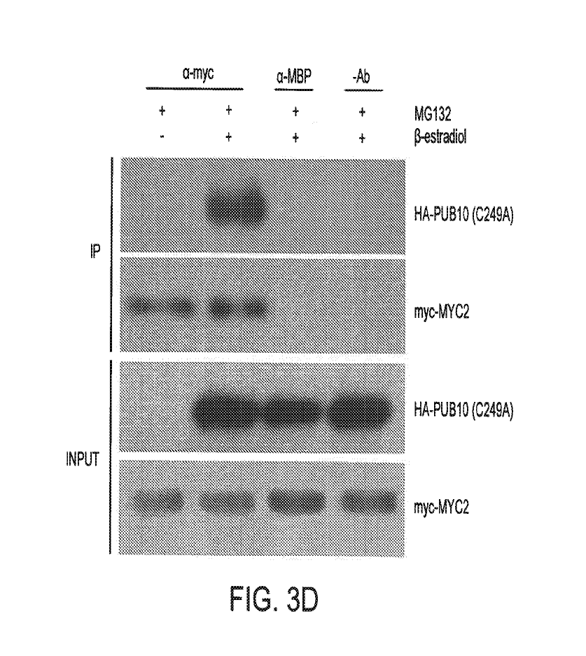

The PUB10/MYC2 interaction in vitro raised the question whether they also do so in vivo. The localization of PUB10-YFP and MYC2-CFP was first examined by transient expression in N. benthaminana leaf cells. PUB10-YFP was found largely localized to plasma membranes and nuclei (FIG. 3A). By contrast, MYC2-CFP was only localized in nuclei (FIG. 3A). The observation that PUB10 and MYC2 were both found in nuclei suggested possible interaction within this organelle. Accordingly, BiFC experiments in N. benthamiana leaf cells were performed. Interestingly, no PUB10-MYC2 interaction was found under normal conditions, but strong interaction was detected only in nuclei following ABA treatment (FIG. 3B). Negative control experiments showed that neither cYFP-PUB10 nor MYC2-nYFP showed any florescence under all conditions investigated.

To see whether PUB10 and MYC2 also interacted in vivo, double-transgenic plants expressing 35S:myc-MYC2 and XVE:HA-PUB10 were generated. Note that the latter is an inducible transgene whose expression requires β-estradiol treatment (Zuo et al., 2000). FIGS. 3C and 3D show that HA-PUB10 and HA-PUB10 (C249A), which were expressed only upon β-estradiol treatment, was pulled down by myc-MYC2. The interaction is clearly specific and dependent on myc-MYC2, as HA-PUB10 was not detected in the non-induced sample. Neither myc-MYC2 nor HA-PUB10 was detected in the absence of an antibody or when anti-MBP antibody was used as a negative control.

### Example 5

### Expression Profile of PUB10-GUS

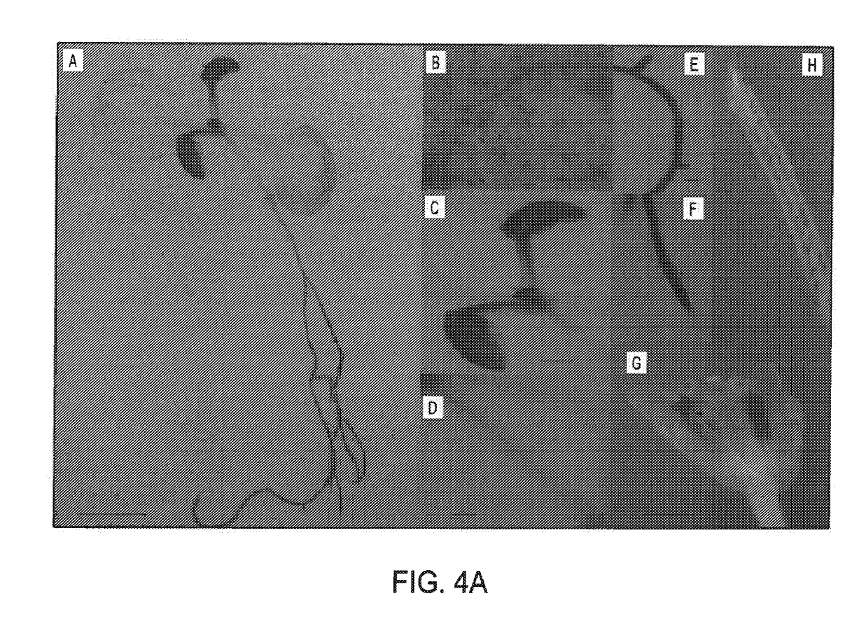

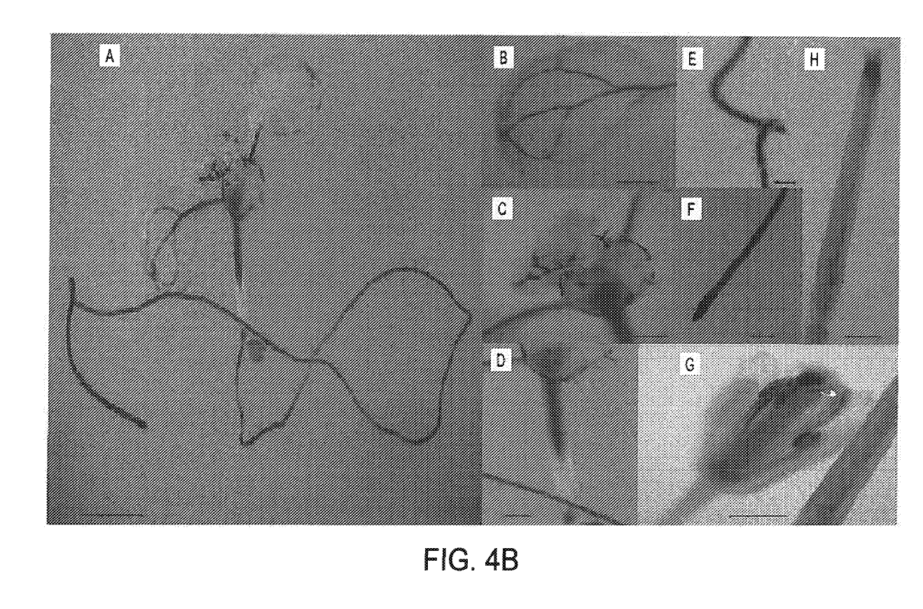

Transgenic plants expressing PUB10 promoter-GUS fusion were generated to determine expression profile of its promoter. FIG. 4A shows that in vegetative tissues PUB10 was strongly expressed in primary and lateral roots, vascular tissues, mesophyll cells and trichomes. Petals, stamen and stigma also showed strong GUS expression and the same was true with embryos. Comparison of the expression profiles of PUB10 and MYC2 showed considerable overlap in tissues and cell types (FIG. 4B). This co-expression result argued that the interaction of the two proteins within the nucleus has physiological relevance.

### Example 6

### Differential Protein Stability of PUB10 and PUB10 (C249A) in Transgenic Plants

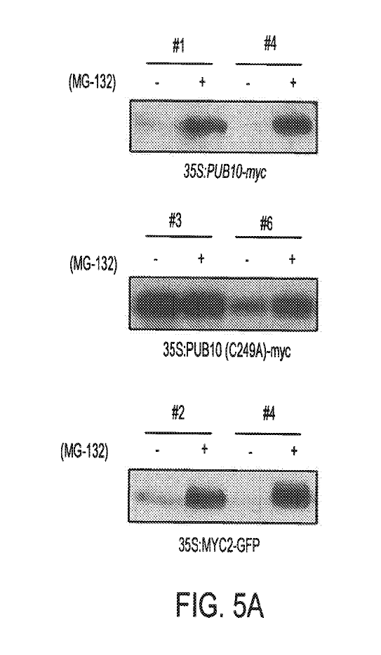

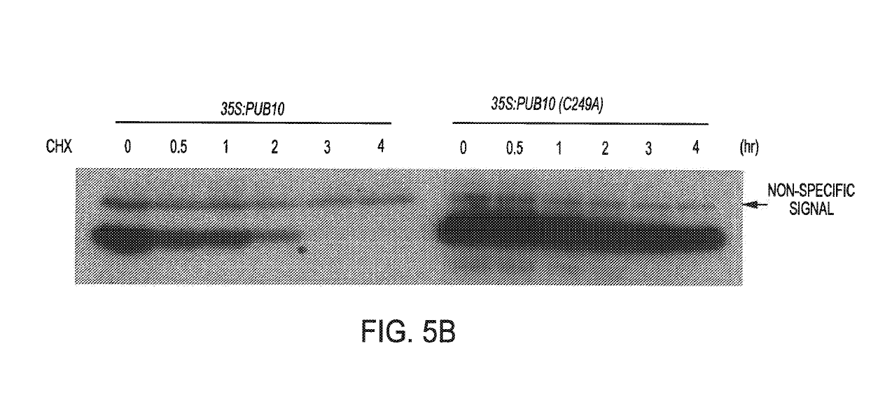

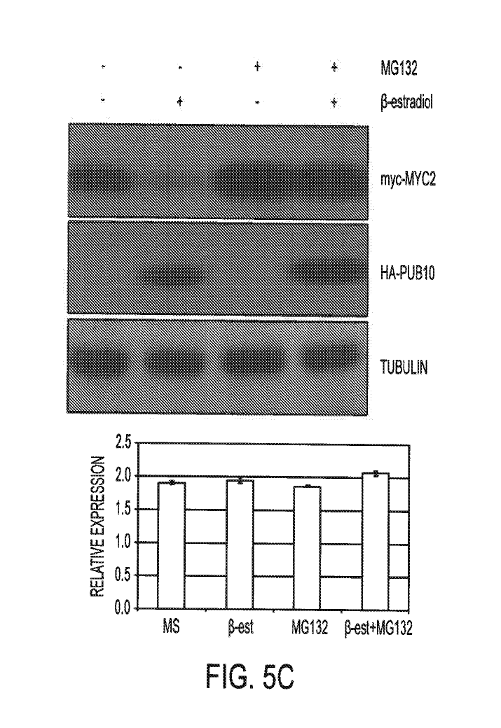

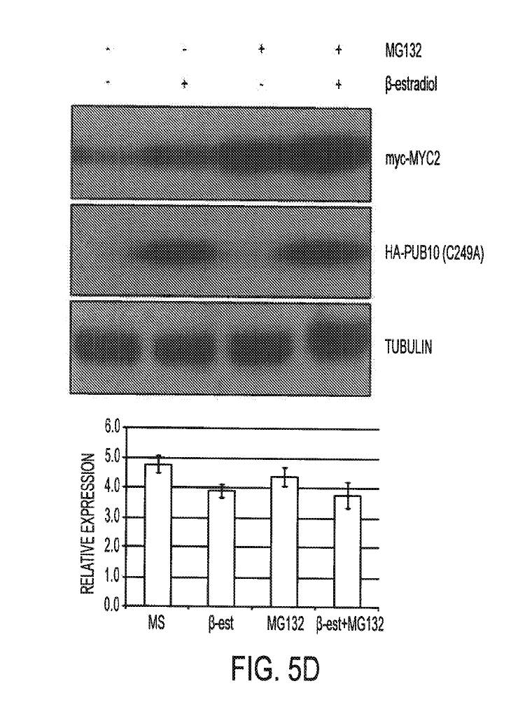

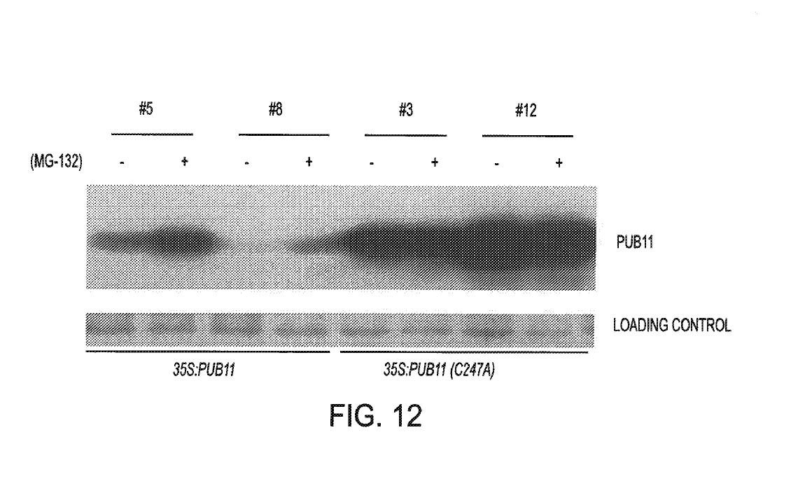

To examine the protein stability of PUB10, PUB10 (C249A) and MYC2 in vivo, cDNAs encoding three proteins by CaMV 35S promoter were overexpressed. Western blot analysis shows that the expression level of wild type PUB10 protein was very low in the non-treated samples, compared with that of PUB10 (C249A) mutant protein (FIG. 5A). A similar result was obtained with MYC2. Because of the extreme instability of PUB10 and MYC2 in vivo, their degradation in transgenic plants was blocked by the 26S proteasome inhibitor, MG132. The protein levels of PUB10 and MYC2 were considerably elevated by the addition of MG132. In contrast, only a moderate increase was observed with PUB10 (C249A) under the same condition (FIG. 5A). Similar results were obtained with PUB11 and PUB11 (C247A) (FIG. 12). From these results, it is concluded that the stability of PUB10 and MYC2 proteins is regulated by 26S proteasome and ubiquitin E3 ligase activity of PUB10 and PUB11 is a major determinant of their stability.

To further confirm self-destruction of PUB10, the time course of PUB10 and PUB10 (C249A) levels was determined after de novo protein synthesis in transgenic seedlings was inhibited by cycloheximide. FIG. 5B shows that wild type PUB10 protein had a half-life of only 1 h, but the half-life of PUB10 (C249A) mutant protein was dramatically prolonged by disruption of the U-box motif.

### Example 7

### Reciprocal Relationship Between PUB10 Activity and MYC2 Protein Levels

The above experiments have shown that PUB10 and MYC2 proteins were expressed in the same cell-types/tissues, form a complex in nuclei and MYC2 was ubiquitinated by PUB10 in vitro. Together, these results suggested that MYC2 may be targeted by PUB10 for ubiquitin-mediated degradation in vivo. To investigate this possibility, two double transgenic plants expressing 35S:myc-MYC2/XVE:HA-PUB10 and 35S:myc-MYC2/XVE:HA-PUB10 (C249A) were generated. Double transgenic plants were treated with β-estradiol alone, MG132 alone or β-estradiol plus MG132 for 16 h. FIGS. 5C and 5D show that induced expression of HA-PUB10 clearly resulted in a decrease in myc-MYC2 level. On the other hand, MYC2 protein level should increase when PUB10 E3 ligase activity is compromised. In contrast to induced expression of PUB10, induced expression of PUB10 (C249A) mutant protein resulted in an increase of MYC2 level. Real time RT-PCR analysis shows that expression of myc-MYC2 transcripts was comparable between treatments. This observation provides evidence that MYC2 is destabilized by PUB10 in vivo.

### Example 8

### Phenotypes of Pub10 Mutant and PUB10 Over-Expression Plants

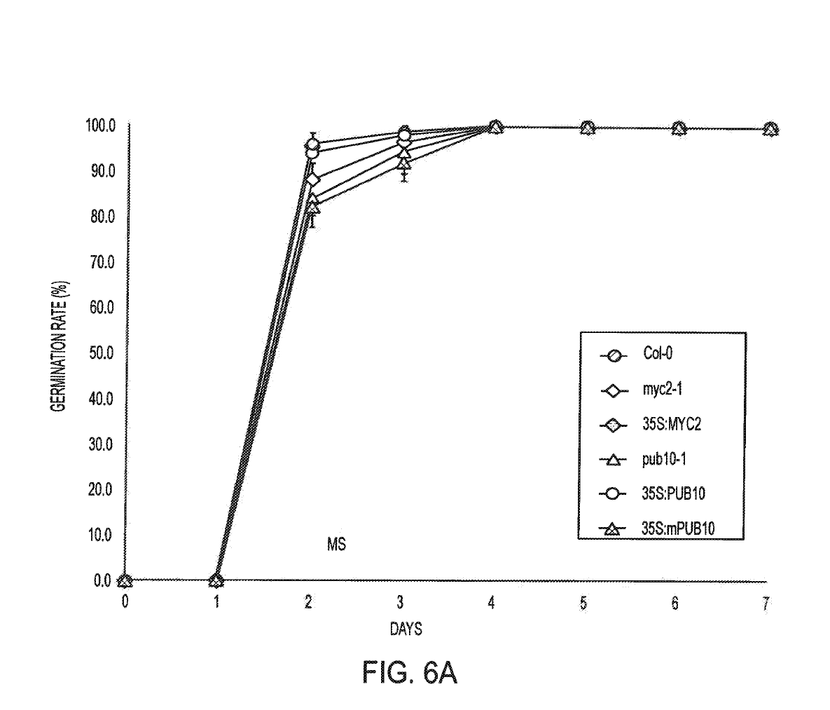

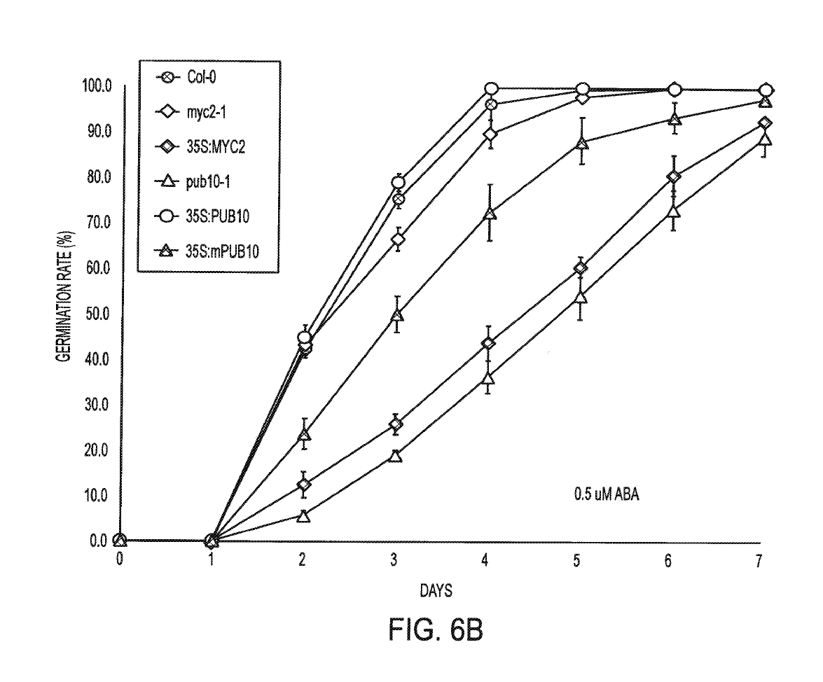

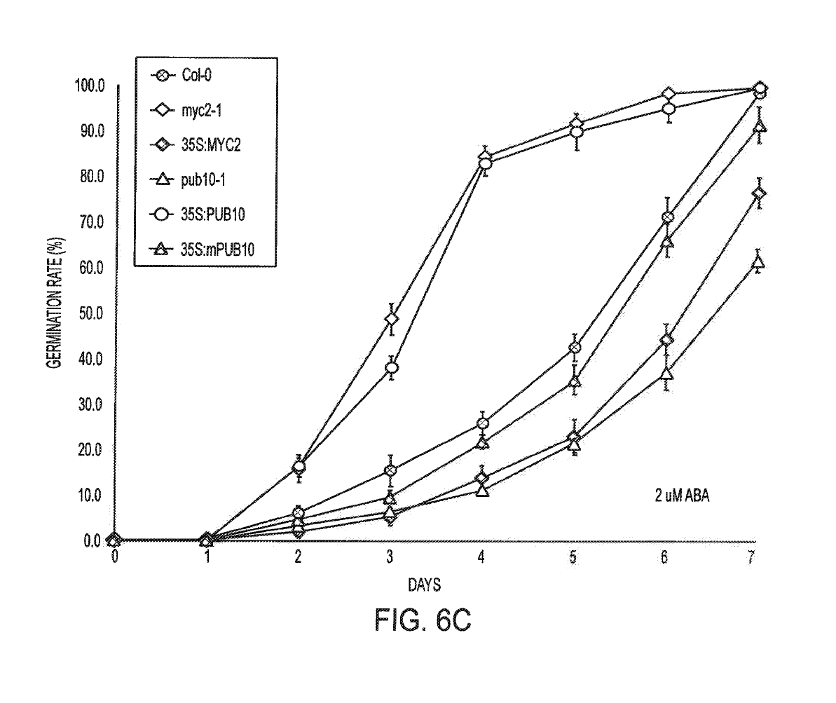

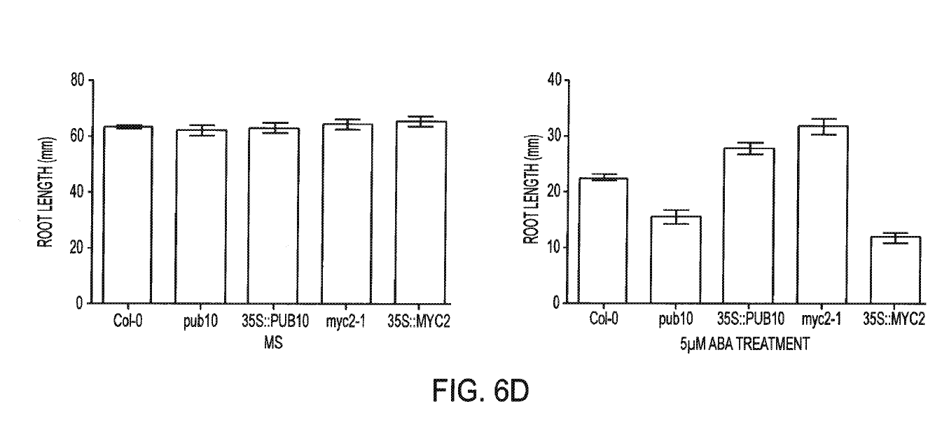

The reciprocal relation between PUB10 activity and MYC2 protein levels suggested the two proteins may play antagonistic roles in signaling pathways known to be regulated by MYC2. MYC2 was first characterized as a positive regulator of ABA signaling pathway and myc2 mutants were hyposensitive/insensitive to ABA during germination (Abe et al., 2003). To investigate ABA phenotypes of PUB 10 deficiency, a T-DNA insertion mutant allele (pub10-1;SALK_017111) was obtained from the SALK collection. In addition, transgenic plants expressing 35S:PUB10 and 35S:PUB10 (C249A) were produced to assess their ABA sensitivity when WT PUB10 or its dominant-negative mutant, respectively, was overexpressed. The ABA sensitivity of WT, mutant and transgenic seed during germination were tested at two different hormone concentrations. It was expected that hypersensitivity would be more apparent at low ABA concentrations and hyposensitivity would be more evident at higher ABA concentration. FIGS. 6A-6D show that seed germination of pub10 mutant alleles was hypersensitive to ABA as compared to WT. These results are consistent with the finding that PUB10 targets MYC2 for degradation in vivo and these mutant alleles should accumulate higher MYC2 levels in vivo. Similar results were seen with 35S:PUB10 (C249A) line which was expected to have reduced MYC2 degradation. In contrast, at this concentration of ABA (0.5 μM) myc2-1 mutant and 35S:PUB10 transgenic line behaved similarly like WT.

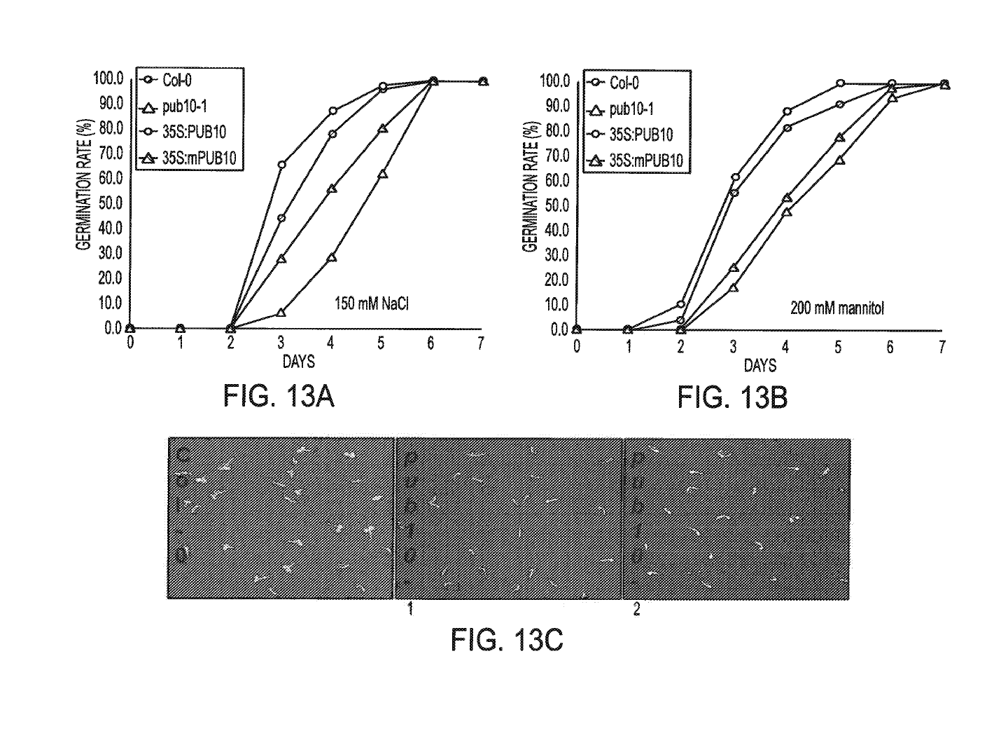

However, clear ABA hyposensitivity was seen with myc2-1 mutant and 35S:PUB10 transgenic lines at 2 μM ABA. Control experiments showed that all of these lines have comparable germination capacity in medium without any ABA (FIGS. 6A-6C). In addition, pub10 mutant and 35S:PUB10 (C249A) showed hypersensitive germination to salt stress (150 mM NaCl) and osmotic stress (200 mM mannitol), whereas 35S:PUB10 showed hyposensitive germination, indicating that PUB10 is a negative regulator in salt and osmotic stress signaling pathways (FIGS. 13A-13C).

In post-germination root elongation assays a similar hypersensitivity of pub10 mutant to ABA (5 uM) was also observed whereas 35S:PUB10 plants were hyposensitive (FIG. 6D). In the same root elongation assays myc2-1 mutant seedlings and 35S:MYC2 transgenic seedlings were hypo-and hypersensitive to ABA, respectively.

### Example 9

### Summary of Experimental Results

No previous work has been done on the two Arabidopsis U-box proteins, PUB10 and 11. The experiments described above show that both proteins exhibited E3 ubiquitin ligase activity with several Arabidopsis UBCs and that the E3 activity was compromised by C249A mutation.

Using PUB10 as a bait to query a small library of transcription factors by yeast two hybrid assays, PUB10 was found to interact with MYC2, a bHLH protein. This interaction in yeast cells was confirmed by in vitro pull-down assays using purified proteins. In addition, by analysis of deletion derivatives, the binding region was localized to the C-terminal fragment of PUB10, which contains the ARM repeats.

The association of PUB10 with MYC2 suggested the latter may be a substrate of the former which was confirmed by in vitro ubiquitination assays. In addition, PUB10 and MYC2 interacted in vivo and their expression profiles in various cell types and organs overlapped to a large extend. Taken together, these results suggested that in vivo MYC2 may be targeted for destruction by PUB10 as a mechanism to terminate signaling. If this was the case, an inverse relationship would be expected between the expression level of PUB10 and that of MYC2. In transgenic plants expressing MYC2, induced expression of PUB10 considerably decreased MYC2 protein level. By contrast, induced expression of PUB10 (C249A) mutant elicited the opposite effect and increased MYC2 protein level. Taken together, these results confirm that PUB10 mediates proteolysis of MYC2 in vivo.

The reciprocal relationship between PUB10 and MYC2 was reflected in their opposing phenotypes in ABA responses. Transgenic plants overexpressing 35S:PUB10 should have low MYC2 levels and should phenocopy myc2 mutant plants. On the other hand, pub10 mutant plants should accumulate more MYC2 and should behave like 35S:MYC2 overexpressing plants. These expected phenotypes were indeed observed when the effects of ABA on root growth were assayed. More important, pub 10 mutant and 35S:PUB10 overexpressing plants have opposite phenotype.

### Example 10

### Transformation of Maize Using Particle Bombardment

Maize plants can be transformed to contain a suppression DNA construct of a validated Arabidopsis lead gene or the corresponding homologs from various species in order to examine the resulting phenotype.

A suppression DNA construct can be cloned into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

The suppression DNA construct can then be introduced into corn cells by particle bombardment. Techniques for corn transformation by particle bombardment have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Suppression DNA constructs that result in a significant delay in wilting or leaf area reduction, yellow color accumulation and/or increased growth rate during drought stress will be considered evidence that the Arabidopsis gene or corresponding homologs functions in maize to enhance drought tolerance.

### Example 11

### Transformation of Maize Using Agrobacterium

Maize plants can be transformed to contain a suppression DNA construct of a validated Arabidopsis lead gene or the corresponding homologs from various species in order to examine the resulting phenotype.

A suppression DNA construct can be cloned into a maize transformation vector. Expression of the gene in the maize transformation vector can be under control of a constitutive promoter such as the maize ubiquitin promoter (Christensen et al., (1989) Plant Mol. Biol. 12:619-632 and Christensen et al., (1992) Plant Mol. Biol. 18:675-689)

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium innoculation, co-cultivation, resting, selection and plant regeneration.

Transgenic T0 plants can be regenerated and their phenotype determined. T1 seed can be collected.

Furthermore, a suppression DNA construct of a validated Arabidopsis gene or homolog thereof can be introduced into an elite maize inbred line either by direct transformation or introgression from a separately transformed line.

### Example 12

### Yield Analysis of Maize Lines with the Arabidopsis Lead Gene

A suppression DNA construct of a validated Arabidopsis gene or homolog thereof can be introduced into an elite maize inbred line either by direct transformation or introgression from a separately transformed line.

Transgenic plants, either inbred or hybrid, can undergo more vigorous field-based experiments to study yield enhancement and/or stability under well-watered and water-limiting conditions.

Subsequent yield analysis can be done to determine whether plants that contain the validated Arabidopsis lead gene have an improvement in yield performance under water-limiting conditions, when compared to the control plants that do not contain the validated Arabidopsis lead gene. Specifically, drought conditions can be imposed during the flowering and/or grain fill period for plants that contain the validated Arabidopsis lead gene and the control plants. Reduction in yield can be measured for both. Plants containing the suppression DNA construct of the Arabidopsis lead gene or homolog thereof have less yield loss relative to the control plants, for example, at least 25%, at least 20%, at least 15%, at least 10% or at least 5% less yield loss.

The above method may be used to select transgenic plants with increased yield, under water-limiting conditions and/or well-watered conditions, when compared to a control plant not comprising said recombinant DNA construct. Plants containing the suppression DNA construct of the Arabidopsis lead gene or homolog thereof may have increased yield, under water-limiting conditions and/or well-watered conditions, relative to the control plants, for example, at least 5%, at least 10%, at least 15%, at least 20% or at least 25% increased yield.

### Example 13

### Transformation of Maize to Contain ZM-PUB10 Suppression DNA Construct

A maize homolog, ZM-PUB10, of the Arabidopsis PUB10 was identified by searching a proprietary database. A nucleotide sequencing encoding ZM-PUB10 is set forth in SEQ ID NO:50 with the corresponding amino acid sequence set forth in SEQ ID NO:51.

A 300 nt fragment of the ZM-PUB10 gene was selected for preparing a suppression DNA construct. The sequence of this fragment is set forth below:

(SEQ ID NO: 52)
gtcagggcgctcgaggctgcccggaggtttgtcgcgctcggacggacgcc
ggccgctgcgggggcgtcagatcaggatgccatctgcaagaatactggtc
ttcagttcaagtatgtgacctggcagttgcaagctgctctggcaaacctg
ccacatagctgttttgagatatcagacgaagttcaagaagaggttgactt
agtgcgagctcagcttagaagagaaatggaaaagaatggaggtcttgatg
taaccgtatttatgaaagttcatgatatcttagctcaaattgacaatgc
t.

Expression of the gene or gene fragment in a maize transformation vector can be under control of a constitutive promoter such as the maize ubiquitin promoter (Christensen et al., 1989, Plant Mol. Biol. 12:619-632 and Christensen et al., 1992, Plant Mol. Biol. 18:675-689).

The maize ubiquitin promoter sequence can be operably linked to the maize ubiquitin intron-1 sequence, or can be operably linked to other intronic sequences. Other introns are known in art that can enhance gene expression, examples of such introns include, but are not limited to: the first intron from Adh1 gene (Callis et al., Genes Dev. 1987 1:1183-1200); and the first intron from Shrunken-1 gene (Mascarenkas et al., Plant Mol. Biol., 1990, 15: 913-920).

A number of plant transcription terminators are known in the art. The terminator from the Sorghum Bicolor gamma-kafirin gene (SB-GKAF) can be used. The sequence of the SB-GKAF terminator is given in WO2013/019461 and is set forth below:

(SEQ ID NO: 53)
aactatctatactgtaataatgttgtatagccgccggatagctagctagt
ttagtcattcagcggcgatgggtaataataaagtgtcatccatccatcac
catgggtggcaacgtgagcaatgacctgattgaacaaattgaaatgaaaa
gaagaaatatgttatatgtcaacgagatttcctcataatgccactgacaa
cgtgtgtccaagaaatgtatcagtgatacgtatattcacaatttttttat
gacttatactcacaatttgtttttttactacttatactcacaatttgttg
tgggtaccataacaatttcgatcgaatatatatcagaaagttgacgaaag
taagctcactcaaaaagttaaatgggctgcggaagctgcgtcaggcccaa
gttttggctattctatccggtatccacgattttgatggctgagggacata
tgttcgctt.

The suppression DNA construct is prepared to have the 300 nt fragment present as an RNAi hairpin (i.e., present in both sense and antisense orientation). The RNAi hairpin is driven by the UB1 promoter. ADH1 intron regulatory cassette and has the SB-GKAF terminator. The suppression DNA construct is introduced into maize cells using either particle bombardment or Agrobacterium-mediated transformation. Transformed plant cells are selected and regenerated to produce transgenic plants having the suppression DNA construct stably integrated into their genome. The transgenic plants are screened for drought tolerance, and drought tolerant plants are obtained.

### Example 14

### Preparation of Soybean Expression Vectors and Transformation of Soybean

Soybean plants can be transformed to suppression DNA construct of a validated Arabidopsis lead gene or the corresponding homologs from various species in order to examine the resulting phenotype.

A suppression DNA construct can be cloned into the PHP27840 vector (PCT Publication No. WO/2012/058528) such that expression of the suppression DNA is under control of the SCP1 promoter (International Publication No. 03/033651).

Soybean embryos may then be transformed with the expression vector. Techniques for soybean transformation and regeneration have been described in International Patent Publication WO 2009/006276, the contents of which are herein incorporated by reference.

T1 plants can be subjected to a soil-based drought stress. Using image analysis, plant area, volume, growth rate and color analysis can be taken at multiple times before and during drought stress. Suppression DNA constructs that result in a significant delay in wilting or leaf area reduction, yellow color accumulation and/or increased growth rate during drought stress will be considered evidence that the Arabidopsis gene or homologs thereof functions in soybean to enhance drought tolerance.

Soybean plants transformed with a suppression DNA construct can then be assayed under more vigorous field-based studies to study yield enhancement and/or stability under well-watered and water-limiting conditions.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Abe, H., Urao, T., Ito, T., Seki, M., Shinozaki, K., and Yamaguchi-Shinozaki, K. (2003). Arabidopsis AtMYC2 (bHLH) and AtMYB2 (MYB) function as transcriptional activators in abscisic acid signaling. Plant Cell 15:63-78.

Andersen, P., Kragelund, B. B., Olsen, A. N., Larsen, F. H., Chua, N. H., Poulsen, F. M., and Skriver, K. (2004). Structure and biochemical function of a prototypical Arabidopsis U-box domain. J. Biol. Chem. 279:40053-40061.

Bergler, J., and Hoth, S. (2011). Plant U-box armadillo repeat proteins AtPUB18 and AtPUB19 are involved in salt inhibition of germination in Arabidopsis. Plant Biol. 13:725-730.

Cho, S. K., Ryu, M. Y., Song, C. Kwak, J. M., and Kim, W. T. (2008). Arabidopsis PUB22 and PUB23 are homologous U-Box E3 ubiquitin ligases that play combinatory roles in response to drought stress. Plant Cell 20:1899-1914.

Kraft, E., Stone, S. L., Ma, L., Su, N., Gao, Y., Lau, O.S., Deng, X. W., and Callis, J. (2005). Genome analysis and functional characterization of the E2 and RING-Type E3 ligase ubiquitination enzymes of Arabidopsis. Plant Physiol. 139:1597-1611.

Lu, D., Lin, W., Gao, X., Wu, S., Cheng, C., Avila, J., Heese, A., Devarenne, T. P., He, P., and Shan, L. (2011). Direct ubiquitination of pattern recognition receptor FLS2 attenuates plant innate immunity. Science 332:1439-1442.

Mitsuda, N., Ikeda, M., Takada, S., Takiguchi, Y., Kondou, Y., Yoshizumi, T., Fujita, M., Shinozaki, K., Matsui, M., and Ohme-Takagi, M. (2010). Efficient yeast one-/two-hybrid screening using a library composed only of transcription factors in Arabidopsis thaliana. Plant Cell Physiol. 51:2145-2151.

Mudgil, Y., Shiu, S. H., Stone, S. L., Salt, J. N., and Goring, D. R. (2004). A large complement of the predicted Arabidopsis ARM repeat proteins are members of the U-box E3 ubiquitin ligase family. Plant Physiol. 134:59-66.

Samuel, M. A., Mudgil, Y., Salt, J. N., Delmas, F., Ramachandran, S., Chilelli, A., and Goring, D. R. (2008). Interactions between the S-domain receptor kinases and AtPUB-ARM E3 ubiquitin ligases suggest a conserved signaling pathway in Arabidopsis. Plant Physiol. 147:2084-2095.

Senecoff, J. F., McKinney, E. C., and Meagher, R. B. (1996). De novo purine synthesis in Arabidopsis thaliana. II. The PUR7 gene encoding 5′-phosphoribosyl-4-(N-succinocarboxamide)-5-aminoimidazole synthetase is expressed in rapidly dividing tissues. Plant Physiol. 112:905-917.

Seo, D. H., Ryu, M. Y., Jammes, F., Hwang, J. H., Turek, M., Kang, B. G., Kwak, J. M., and Kim, W. T. (2012). Roles of four Arabidopsis U-box E3 ubiquitin ligases in negative regulation of abscisic acid-mediated drought stress responses. Plant Physiol. 160:556-568.

Trujillo, M., Ichimura, K., Casais, C., and Shirasu, K. (2008). Negative regulation of PAMP-triggered immunity by an E3 ubiquitin ligase triplet in Arabidopsis. Curr. Biol. 2008 18:1396-1401.

Valliyodan, B., and Nguyen, H. T., (2006). Understanding regulatory networks and engineering for enhanced drought tolerance in plants. Curr. Opin. Plant Biol. 9:189-195.

Vierstra, R. D. (2009). The ubiquitin-26S proteasome system at the nexus of plant biology. Nat. Rev. Mol. Cell Biol. 10: 385-397.

Wiborg, J., O'Shea, C., and Skriver, K. (2008). Biochemical function of typical and variant Arabidopsis thaliana U-box E3 ubiquitin-protein ligases. Biochem J. 413:447-457.

Yang, C. W., González-Lamothe, R., Ewan, R. A., Rowland, O., Yoshioka, H., Shenton, M., Ye, H., O'Donnell, E., Jones, J. D., and Sadanandom, A. (2006). The E3 ubiquitin ligase activity of Arabidopsis PLANT U-BOX17 and its functional tobacco homolog ACRE276 are required for cell death and defense. Plant Cell 18:1084-1098.

Yee, D., and Goring, D. R. (2009). The diversity of plant U-box E3 ubiquitin ligases: from upstream activators to downstream target substrates. J. Exp. Bot. 60:1109-1121.

Zhang, X., Henriques, R., Lin, S. S., Niu, Q. W., and Chua, N. H. (2006). Agrobacterium-mediated transformation of Arabidopsis thaliana using the floral dip method. Nat. Protocol 1:641-646.

Zuo, J., Niu, Q. W., and Chua, N. H. (2000). Technical advance: An estrogen receptor-based transactivator XVE mediates highly inducible gene expression in transgenic plants. Plant J. 24:265-273.

1. A plant comprising in its genome a suppression DNA construct comprising at least one heterologous regulatory element operably linked to all or part of a polynucleotide, wherein said polynucleotide is selected from the group consisting of:
(a) a polynucleotide comprising a nucleotide sequence encoding a polypeptide, wherein the polypeptide has an amino acid sequence of at least 90% sequence identity, based on the Clustal V or Clustal W method of alignment, when compared to SEQ ID NO:2 or 51;
(b) a polynucleotide comprising a nucleotide sequence encoding a polypeptide wherein the amino acid sequence of the polypeptide comprises SEQ ID NO:2 or 51;
(c) a polynucleotide comprising a region derived from all or part of a sense strand or antisense strand of a target gene of interest, said region comprising a nucleotide sequence of at least 90% sequence identity, based on the Clustal V or Clustal W method of alignment, when compared to all or part of a sense strand or antisense strand from which said region is derived, wherein the target gene of interest encodes a PUB10 polypeptide;
(d) a polynucleotide comprising a nucleotide sequence of at least 90% sequence identity, based on the Clustal V or Clustal W method of alignment, when compared to SEQ ID NO:1 or 50; and
(e) a polynucleotide comprising a nucleotide sequence comprising SEQ ID NO:1 or 50;
(f) a polynucleotide comprising a nucleotide sequence hybridizable under stringent conditions with a DNA molecule comprising the full complement of SEQ ID NO:1 or 50;
(g) a polynucleotide comprising a nucleotide sequence derived from SEQ ID NO:1 or 50 by alteration of one or more nucleotides by at least one method selected from the group consisting of: deletion, substitution, addition and insertion; and
(h) a modified plant miRNA precursor, wherein the precursor has been modified to replace the miRNA encoding region with a sequence designed to produce an miRNA directed to SEQ ID NO:1 or 50;
and wherein said plant exhibits increased drought tolerance when compared to a control plant not comprising said suppression DNA construct.

2. The plant of claim 1, wherein the plant is a monocot or dicot.

3. The plant of claim 3 wherein the plant is selected from the group consisting of: maize, soybean, sunflower, sorghum, canola, wheat, alfalfa, cotton, rice, barley, millet, sugarcane, switchgrass, tobacco, potato and sugar beet.

4. A seed of the plant of claim 1, wherein said seed comprises in its genome said suppression construct and wherein a plant produced from said seed exhibits drought tolerance.

5. A plant comprising in its genome a suppression DNA construct comprising at least one heterologous regulatory element operably linked to all or part of a polynucleotide, wherein said polynucleotide is selected from the group consisting of:
(a) a polynucleotide comprising a nucleotide sequence encoding a polypeptide, wherein the polypeptide has an amino acid sequence of at least 90% sequence identity, based on the Clustal V or Clustal W method of alignment, when compared to SEQ ID NO:2 or 51;
(b) a polynucleotide comprising a nucleotide sequence encoding a polypeptide wherein the amino acid sequence of the polypeptide comprises SEQ ID NO:2 or 51;
(c) a polynucleotide comprising a region derived from all or part of a sense strand or antisense strand of a target gene of interest, said region comprising a nucleotide sequence of at least 90% sequence identity, based on the Clustal V or Clustal W method of alignment, when compared to all or part of a sense strand or antisense strand from which said region is derived, wherein the target gene of interest encodes a PUB10 polypeptide;
(d) a polynucleotide comprising a nucleotide sequence of at least 90% sequence identity, based on the Clustal V or Clustal W method of alignment, when compared to SEQ ID NO:1 or 50; and
(e) a polynucleotide comprising a nucleotide sequence comprising SEQ ID NO:1 or 50;
(f) a polynucleotide comprising a nucleotide sequence hybridizable under stringent conditions with a DNA molecule comprising the full complement of SEQ ID NO:1 or 50;
(g) a polynucleotide comprising a nucleotide sequence derived from SEQ ID NO:1 or 50 by alteration of one or more nucleotides by at least one method selected from the group consisting of: deletion, substitution, addition and insertion; and
(h) a modified plant miRNA precursor, wherein the precursor has been modified to replace the miRNA encoding region with a sequence designed to produce an miRNA directed to SEQ ID NO:1 or 50;
and wherein said plant exhibits an increase in yield when compared to a control plant not comprising said suppression DNA construct.

6. The plant of claim 5, wherein said plant exhibits said increase in yield when compared, under water limiting conditions, to said control plant not comprising said suppression DNA construct.

7. The plant of claim 5, wherein the plant is a monocot or dicot.

8. The plant of claim 7 wherein the plant is selected from the group consisting of: maize, soybean, sunflower, sorghum, canola, wheat, alfalfa, cotton, rice, barley, millet, sugarcane, switchgrass, tobacco, potato and sugar beet.

9. A seed of the plant of claim 5, wherein said seed comprises in its genome said suppression construct and wherein a plant produced from said seed exhibits increased yield.

10. A method of increasing drought tolerance in a plant, comprising:
(a) introducing into a regenerable plant cell a suppression DNA construct comprising at least one heterologous regulatory element operably linked to all or part of a polynucleotide, wherein said polynucleotide is selected from the group consisting of:
(i) a polynucleotide comprising a nucleotide sequence encoding a polypeptide, wherein the polypeptide has an amino acid sequence of at least 90% sequence identity, based on the Clustal V or Clustal W method of alignment, when compared to SEQ ID NO:2 or 51;
(ii) a polynucleotide comprising a nucleotide sequence encoding a polypeptide wherein the amino acid sequence of the polypeptide comprises SEQ ID NO:2 or 51;
(iii) a polynucleotide comprising a region derived from all or part of a sense strand or antisense strand of a target gene of interest, said region comprising a nucleotide sequence of at least 90% sequence identity, based on the Clustal V or Clustal W method of alignment, when compared to all or part of a sense strand or antisense strand from which said region is derived, wherein the target gene of interest encodes a PUB10 polypeptide;
(iv) a polynucleotide comprising a nucleotide sequence of at least 90% sequence identity, based on the Clustal V or Clustal W method of alignment, when compared to SEQ ID NO:1 or 50;
(v) a polynucleotide comprising a nucleotide sequence comprising SEQ ID NO:1 or 50;
(vi) a polynucleotide comprising a nucleotide sequence hybridizable under stringent conditions with a DNA molecule comprising the full complement of SEQ ID NO:1 or 50;
(vii) a polynucleotide comprising a nucleotide sequence derived from SEQ ID NO:1 or 50 by alteration of one or more nucleotides by at least one method selected from the group consisting of: deletion, substitution, addition and insertion; and
(viii) a modified plant miRNA precursor, wherein the precursor has been modified to replace the miRNA encoding region with a sequence designed to produce an miRNA directed to SEQ ID NO:1 or 50;
(b) regenerating a transgenic plant from the regenerable plant cell after step (a), wherein the transgenic plant comprises in its genome the recombinant DNA construct and exhibits increased drought tolerance when compared to a control plant not comprising the recombinant DNA construct.

11. The method of claim 10, further comprising:
(c) obtaining a progeny plant derived from the transgenic plant, wherein said progeny plant comprises in its genome the suppression DNA construct and exhibits increased drought tolerance when compared to a control plant not comprising the suppression DNA construct.

12. A method of selecting for (or identifying) increased drought tolerance in a plant, comprising:
(a) obtaining a transgenic plant, wherein the transgenic plant comprises in its genome a suppression DNA construct comprising at least one heterologous regulatory element operably linked to all or part of a polynucleotide, wherein said polynucleotide is selected from the group consisting of:
(i) a polynucleotide comprising a nucleotide sequence encoding a polypeptide, wherein the polypeptide has an amino acid sequence of at least 90% sequence identity, based on the Clustal V or Clustal W method of alignment, when compared to SEQ ID NO:2 or 51;
(ii) a polynucleotide comprising a nucleotide sequence encoding a polypeptide wherein the amino acid sequence of the polypeptide comprises SEQ ID NO:2 or 51;
(iii) a polynucleotide comprising a region derived from all or part of a sense strand or antisense strand of a target gene of interest, said region comprising a nucleotide sequence of at least 90% sequence identity, based on the Clustal V or Clustal W method of alignment, when compared to all or part of a sense strand or antisense strand from which said region is derived, wherein the target gene of interest encodes a PUB10 polypeptide;
(iv) a polynucleotide comprising a nucleotide sequence of at least 90% sequence identity, based on the Clustal V or Clustal W method of alignment, when compared to SEQ ID NO:1 or 50;
(v) a polynucleotide comprising a nucleotide sequence comprising SEQ ID NO:1 or 50;
(vi) a polynucleotide comprising a nucleotide sequence hybridizable under stringent conditions with a DNA molecule comprising the full complement of SEQ ID NO:1 or 50;
(vii) a polynucleotide comprising a nucleotide sequence derived from SEQ ID NO:1 or 50 by alteration of one or more nucleotides by at least one method selected from the group consisting of: deletion, substitution, addition and insertion; and
(viii) a modified plant miRNA precursor, wherein the precursor has been modified to replace the miRNA encoding region with a sequence designed to produce an miRNA directed to SEQ ID NO:1 or 50;
(b) obtaining a progeny plant derived from the transgenic plant of (a), wherein the progeny plant comprises in its genome the suppression DNA construct; and
(c) selecting (or identifying) the progeny plant with increased drought tolerance compared to a control plant not comprising the suppression DNA construct.

13. A method of selecting for (or identifying) an alteration of an agronomic characteristic in a plant, comprising:
(a) obtaining a transgenic plant, wherein the transgenic plant comprises in its genome a suppression DNA construct comprising at least one heterologous regulatory element operably linked to all or part of a polynucleotide, wherein said polynucleotide is selected from the group consisting of:
(i) a polynucleotide comprising a nucleotide sequence encoding a polypeptide, wherein the polypeptide has an amino acid sequence of at least 90% sequence identity, based on the Clustal V or Clustal W method of alignment, when compared to SEQ ID NO:2 or 51;
(ii) a polynucleotide comprising a nucleotide sequence encoding a polypeptide wherein the amino acid sequence of the polypeptide comprises SEQ ID NO:2 or 51;
(iii) a polynucleotide comprising a region derived from all or part of a sense strand or antisense strand of a target gene of interest, said region comprising a nucleotide sequence of at least 90% sequence identity, based on the Clustal V or Clustal W method of alignment, when compared to all or part of a sense strand or antisense strand from which said region is derived, wherein the target gene of interest encodes a PUB10 polypeptide;
(iv) a polynucleotide comprising a nucleotide sequence of at least 90% sequence identity, based on the Clustal V or Clustal W method of alignment, when compared to SEQ ID NO:1 or 50;
(v) a polynucleotide comprising a nucleotide sequence comprising SEQ ID NO:1 or 50;
(vi) a polynucleotide comprising a nucleotide sequence hybridizable under stringent conditions with a DNA molecule comprising the full complement of SEQ ID NO:1 or 50;
(vii) a polynucleotide comprising a nucleotide sequence derived from SEQ ID NO:1 or 50 by alteration of one or more nucleotides by at least one method selected from the group consisting of: deletion, substitution, addition and insertion; and
(viii) a modified plant miRNA precursor, wherein the precursor has been modified to replace the miRNA encoding region with a sequence designed to produce an miRNA directed to SEQ ID NO:1 or 50;
(b) obtaining a progeny plant derived from the transgenic plant of (a), wherein the progeny plant comprises in its genome the suppression DNA construct; and
(c) selecting (or identifying) the progeny plant which exhibits an alteration of at least one agronomic characteristic when compared to a control plant not comprising the suppression DNA construct.

14. The method of claim 13, wherein said at least one agronomic trait is yield and further wherein said alteration is an increase.

15. The method of claim 13, wherein said step (c) comprises determining whether the transgenic plant exhibits an alteration of at least one agronomic characteristic when compared, under water limiting conditions, to a control plant not comprising the suppression DNA construct.

16. The method of claim 10, wherein the plant is a monocot or a dicot.

17. The method of claim 16, wherein the plant is selected from the group consisting of: maize, soybean, sunflower, sorghum, canola, wheat, alfalfa, cotton, rice, barley, millet, sugarcane, switchgrass, tobacco, potato and sugar beet.

18. The plant of claim 1, wherein the suppression DNA construct includes a nucleotide sequence set forth in SEQ ID NO:52.

19. The seed of claim 4, wherein the suppression DNA construct includes a nucleotide sequence set forth in SEQ ID NO:52.

20. The method of claim 10, wherein the suppression DNA construct includes a nucleotide sequence set forth in SEQ ID NO:52.

21. A seed of the plant of claim 6, wherein said seed comprises in its genome said suppression construct and wherein a plant produced from said seed exhibits increased yield.

22. The method of claim 14, wherein said step (c) comprises determining whether the transgenic plant exhibits an alteration of at least one agronomic characteristic when compared, under water limiting conditions, to a control plant not comprising the suppression DNA construct.

23. The plant of claim 5, wherein the suppression DNA construct includes a nucleotide sequence set forth in SEQ ID NO:52.

24. The plant of claim 6, wherein the suppression DNA construct includes a nucleotide sequence set forth in SEQ ID NO:52.

25. The seed of claim 9, wherein the suppression DNA construct includes a nucleotide sequence set forth in SEQ ID NO:52.
